(19) United States
(12) Patent Application Publication
(10) Pub. No.: US 2009/0312370 A1
(43) Pub. Date: Dec. 17, 2009

(54) MACROCYCLIC COMPOUNDS USEFUL AS BACE INHIBITORS

(76) Inventors: Kurt Laumen, March (DE); Rainer Machauer, Freiburg (DE); Marina Tintelnot-Blomley, Maulburg (DE); Siem Jacob Veenstra, Lorrach (DE)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/374,467

(22) PCT Filed: Jul. 20, 2007
(86) PCT No.: PCT/EP07/57540

§ 371 Date: Jan. 20, 2009

(30) Foreign Application Priority Data
Jul. 20, 2006 (EP) 06117571.7

(51) Int. Cl. A61K 31/4439; C07D 211/70; A61K 31/44; C07D 413/12

(52) U.S. Cl. 514/340; 546/337; 514/357; 546/271.4

(57) ABSTRACT

The invention relates to novel macrocyclic compounds of the formula

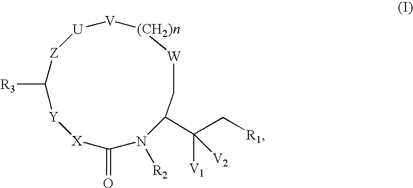

in which all of the variables are as defined in the specification, in free base form or in acid addition salt form, to their preparation, to their use as medicaments and to medicaments comprising them.

The present invention relates to novel macrocyclic compounds, to their preparation, to their use as medicaments and to medicaments comprising them.

More particularly, the invention relates to a compound of the formula

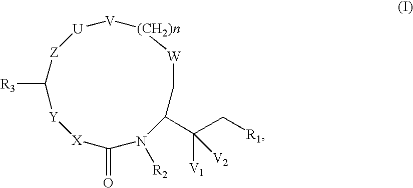

in which
R₁ is —(CH₂)ₖN(Rₐ)R_{b}, in which
   k is 0, 1 or 2;
   Rₐ is hydrogen or an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydroquinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group; and
   R_{b} is a (C₃₋₈)cycloalkyl group, in which
      (a) one of the carbon ring members of the (C₃₋₈)cycloalkyl moiety, which are different from the carbon ring member, to which the nitrogen atom carrying Rₐ is attached, is optionally replaced by a hetero ring member, selected from the group consisting of —O—, —S—, —S(═O)—, —S(═O)₂— and —N(R_{c})—, in which
         R_{c} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group,
      (b) the (C₃₋₈)cycloalkyl moiety is substituted by 1 to 4 substituents, independently selected from the group consisting of halogen, cyano, oxo, hydroxy, (C₁₋₄)-alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, non-aromatic heterocyclyl, non-aromatic heterocyclyl (C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]-oxepin-5-yl group, and
      (c) the (C₃₋₈)cycloalkyl moiety is optionally substituted at two adjacent carbon ring members by two substituents, which form, together with the two adjacent carbon ring members, to which they are attached, a (C₃₋₈)cycloalkyl group, in which
         (i) one of the carbon ring members of the (C₃₋₈)cycloalkyl group thus formed, which are different from the said two adjacent carbon ring members, to which the said two substituents are optionally attached, is optionally replaced by a hetero ring member, selected from the group consisting of —O—, —S—, —S(═O)—, —S(═O)₂— and —N(R_{d})—, in which
            R_{d} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group, and
         (ii) the (C₃₋₈)cycloalkyl group thus formed is optionally substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]-oxathiin-4-yl, 2,3,4,5-tetrahydro-benzo[b]oxepin-5-yl or 1,3,4,5-tetrahydro-benzo[c]-oxepin-5-yl group;
R₂ is hydrogen or (C₁₋₈)alkyl;
R₃ is hydrogen, (C₁₋₈)alkyl or an optionally substituted (C₁₋₈)alkylOC(═O)NH, (C₃₋₈)cycloalkylOC(═O)NH, (C₃₋₈)cycloalkyl(C₁₋₄)alkylOC(═O)NH, aryl(C₁₋₄)alkylOC(═O)NH, heteroaryl(C₁₋₄)alkylOC(═O)NH, (C₁₋₄)alkylC(═O)NH, (C₃₋₈)cycloalkylC(═O)NH, arylC(═O)NH, aryl(C₁₋₄)alkylC(═O)NH, heteroarylC(═O)NH or heteroaryl(C₁₋₄)alkylC(═O)NH group;
U is a bond, CF₂, CF₂CF₂, CHF, CHFCHF, cycloprop-1,2-ylene, (C₁₋₃)alkylenoxy, (C₁₋₃)alkylenamino, (C₁₋₈)alkylene, NRₑ or an aromatic or heteroaromatic ring, which ring is optionally substituted with halogen, (C₁₋₈)alkoxy, hydroxy or (C₁₋₈)alkyl, whereby Z and V are in ortho- or meta-position to each other, wherein
   Rₑ is hydrogen, (C₁₋₈)alkyl or (C₃₋₇)cycloalkyl;
V is CH═CH, cycloprop-1,2-ylene, CH₂CH(OH), CH(OH)CH₂ or CR_{f}R_{f}CR_{f}R_{f}, wherein each R_{f}, independently, is hydrogen, fluorine or (C₁₋₈)alkyl;
   either
V₁ is hydrogen and
V₂ is hydroxy
   or
V₁ and V₂ together are oxo;
W is (C₁₋₈)alkylene, O, S, S(═O)₂, C(═O), C(═O)O, OC(═O), N(R_{g})C(═O), C(═O)NR_{g} or NR_{g}, wherein
   R_{g} is hydrogen or (C₁₋₈)alkyl;
X is an optionally substituted aromatic or heteroaromatic ring, whereby Y and C(═O)NR₂ are in meta-position to each other;
Y is a bond, O, S(═O)₂, S(═O)₂NRₕ, N(Rₕ)S(═O)₂, NRₕ, C(Rₕ)OH, C(═O)NRₕ, N(Rₕ)C(═O), C(═O)N(Rₕ)O or ON(Rₕ)C(═O), wherein
   Rₕ is hydrogen, (C₁₋₈)alkyl or (C₃₋₈)cycloalkyl;
Z is O, CH₂, CF₂, CHF, CH═CH, cycloprop-1,2-ylene or a bond; and
n is 0 to 5,
   the number of ring atoms included in the macrocyclic ring being 14, 15, 16 or 17, in free base form or in acid addition salt form.

E. g. on account of one or more than one asymmetrical carbon atom, which may be present in a compound of the formula I, a corresponding compound of the formula I may exist in pure optically active form or in the form of a mixture of optical isomers, e.g. in the form of a racemic mixture. All of such pure optical isomers and all of their mixtures, including the racemic mixtures, are part of the present invention.

A compound of the formula I may exist in free base form or in acid addition salt form. All of such free compounds and salts are part of the present invention.

A compound of the formula I may exist in tautomeric form. All of such tautomers are part of the present invention.

Halogen denotes fluorine, chlorine, bromine or iodine.

Optional substituents on alkyl, cycloalkyl or non-aromatic heterocyclyl groups or moieties may be one to four groups independently selected from hydroxy, hydroxy(C₁₋₄)alkyl, (C₁₋₄)-alkoxy, (C₁₋₄)alkoxy(C₁₋₄)alkyl, (C₁₋₄)alkoxy(C₁₋₄)alkoxy, (C₁₋₄)alkylsulfanyl, (C₁₋₄)alkoxycarbonyl, (C₁₋₄)alkylcarbonyloxy, (C₁₋₄)alkylcarbonyl, (C₁₋₄)alkylsulfonyl, cyano, oxo, (C₃₋₇)cycloalkyl, optionally substituted aryl, optionally substituted aryl(C₁₋₄)alkyl, optionally substituted heteroaryl and optionally substituted heteroaryl(C₁₋₄)alkyl.

Optional substituents on chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydroquinol-4-yl, 1,2,3,4-tetrahydroisoquinol-4-yl, 1,2,3,4-tetrahydronaphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c]-[1,2]oxathiin-4-yl, 2,2-dioxo-3,4-dihydro-2H-2lambda*6*-benzo[e][1,2]oxathiin-4-yl, 2,3,4,5-tetrahydrobenzo[b]oxepin-5-yl, 1,3,4,5-tetrahydrobenzo[c]oxepin-5-yl, aryl or heteroaryl groups or moieties or on aromatic or heteroaromatic rings may be one to four, especially one to three, groups independently selected from hydroxy, (C₁₋₈)alkyl, (C₁₋₆)alkoxy, (C₁₋₄)alkoxy-(C₁₋₄)alkyl, S(═O)₂(C₁₋₄)alkyl, (C₃₋₇)cycloalkyl, (C₃₋₇)cycloalkyl(C₁₋₄)alkyl, cyano, nitro, trifluoromethyl, halogen, optionally substituted aryl, optionally substituted heteroaryl and optionally substituted carbamoyl.

An optionally substituted aryl or heteroaryl group or moiety or an aromatic or heteroaromatic ring may also carry, as optional substituents, one to three groups selected from benzyloxy, phenoxy, S(═O)₂NH₂, N(H)S(═O)₂(C₁₋₃)alkyl, carboxy, (C₁₋₄)alkoxycarbonyl, (C₁₋₄)alkylcarbamoyl, (C₁₋₄)alkylcarbonyloxy, (C₁₋₄)alkylcarbonyl, hydroxy(C₁₋₄)alkyl and optionally substituted amino.

Optional substituents on amino groups or moieties can be one or two groups independently selected from (C₁₋₄)alkyl, (C₁₋₄)alkoxy(C₁₋₄)alkyl, (C₁₋₄)alkoxycarbonyl, aryl(C₁₋₄)alkoxycarbonyl and heteroaryl(C₁₋₄)alkoxycarbonyl.

Optional substituents on carbamoyl groups or moieties can be one or two groups selected from (C₁₋₄)alkyl and (C₁₋₄)alkoxy(C₁₋₄)alkyl.

Aryl or an aromatic ring is naphthyl or preferably phenyl. It can also be fused with a cycloalkyl or a heteroaromatic ring (e.g. to form a quinolyl or indolyl group).

Heteroaryl or a heteroaromatic ring is an aromatic 5- or 6-membered ring, in which 1, 2 or 3 ring atoms are hetero atoms independently selected from O, N and S, such as thiazolyl, pyrimidyl or, preferably, oxazolyl, isoxazolyl or pyridyl. It can also be fused with a cycloalkyl or an aromatic or heteroaromatic ring (e.g. to form a quinolyl or indolyl group).

A non-aromatic heterocyclyl group or moiety is a non-aromatic 5- or 6-membered cyclic structure, in which cyclic structure 1, 2 or 3 ring members are hetero ring members independently selected from the group, consisting of a nitrogen ring member, an oxygen ring member and a sulfur ring member, such as pyrrolinyl, pyrrolidyl, tetrahydrofuryl, tetrahydrothienyl, piperidyl, piperazinyl, tetrahydropyranyl or morpholinyl.

Any non-cyclic carbon containing group or moiety with more than 1 carbon atom is straight-chain or branched.

Unless defined otherwise, carbon containing groups, moieties or molecules contain 1 to 8, preferably 1 to 6, preferably 1 to 4, preferably 1 or 2, carbon atoms.

In preferred embodiments, the invention relates to a compound of the formula I, in free base form or in acid addition salt form, in which

(1) R₁ is —(CH₂)ₖN(Rₐ)R_{b}, in which
k is 0, 1 or 2;
Rₐ is hydrogen or an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydroquinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group; and
R_{b} is a (C₃₋₈)cycloalkyl group, in which
   (a) one of the carbon ring members of the (C₃₋₈)cycloalkyl moiety, which are different from the carbon ring member, to which the nitrogen atom carrying Rₐ is attached, is optionally replaced by a hetero ring member, selected from the group consisting of —O—, —S—, —S(═O)—, —S(═O)₂— and —N(R_{c})—, in which
      R_{c} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group,
   (b) the (C₃₋₈)cycloalkyl moiety is substituted by 1 to 4 substituents, independently selected from the group consisting of halogen, cyano, oxo, hydroxy, (C₁₋₄)-alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, non-aromatic heterocyclyl, non-aromatic heterocyclyl (C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]-oxepin-5-yl group, and
   (c) the (C₃₋₈)cycloalkyl moiety is optionally substituted at two adjacent carbon ring members by two substituents, which form, together with the two adjacent carbon ring members, to which they are attached, a (C₃₋₈)cycloalkyl group, in which
      (i) one of the carbon ring members of the (C₃₋₈)cycloalkyl group thus formed, which are different from the said two adjacent carbon ring members, to which the said two substituents are optionally attached, is optionally replaced by a hetero ring member, selected from the group consisting of —O—, —S—, —S(═O)—, —S(═O)₂— and —N(R_{d})—, in which
         R_{d} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group, and
      (ii) the (C₃₋₈)cycloalkyl group thus formed is optionally substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]-oxathiin-4-yl, 2,3,4,5-tetrahydro-benzo[b]oxepin-5-yl or 1,3,4,5-tetrahydro-benzo[c]-oxepin-5-yl group;
         preferably —(CH₂)ₖN(Rₐ)R_{b}, in which
k is 0;
Rₐ is hydrogen; and
R_{b} is a (C₃₋₈)cycloalkyl group, which (C₃₋₈)cycloalkyl group is substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydrobenzo[c]oxepin-5-yl group;
   preferably —(CH₂)ₖN(Rₐ)R_{b}, in which
k is 0;
Rₐ is hydrogen; and
R_{b} is a (C₃₋₈)cycloalkyl group, which (C₃₋₈)cycloalkyl group is mono-substituted by an optionally substituted aryl or heteroaryl group;
   preferably —(CH₂)ₖN(Rₐ)R_{b}, in which
k is 0;
Rₐ is hydrogen; and
R_{b} is a (C₃₋₈)cycloalkyl group, which (C₃₋₈)cycloalkyl group is mono-substituted by an optionally substituted phenyl, pyridyl or isoxazolyl group;
   preferably —(CH₂)ₖN(Rₐ)R_{b}, in which
k is 0;
Rₐ is hydrogen; and
R_{b} is a (C₃₋₈)cycloalkyl group, which (C₃₋₈)cycloalkyl group is mono-substituted by a phenyl, pyridyl or isoxazolyl group, which phenyl, pyridyl or isoxazolyl group is mono-substituted by halogen or (C₁₋₈)alkyl;
   preferably —(CH₂)ₖN(Rₐ)R_{b}, in which
k is 0;
Rₐ is hydrogen; and
R_{b} is a (C₃₋₆)cycloalkyl group, which (C₃₋₆)cycloalkyl group is mono-substituted, preferably in the 1-position, by a phenyl, pyridyl or isoxazolyl group, which phenyl, pyridyl or isoxazolyl group is mono-substituted by halogen or (C₁₋₇)alkyl;
   preferably —(CH₂)ₖN(Rₐ)R_{b}, in which
k is 0;
Rₐ is hydrogen; and
R_{b} is a cyclopropyl group, which cyclopropyl group is mono-substituted, preferably in the 1-position, by a phenyl, pyridyl or isoxazolyl group, which phenyl, pyridyl or isoxazolyl group is mono-substituted by halogen or (C₁₋₆)alkyl;
   (2) R₂ is hydrogen or (C₁₋₈)alkyl;
   preferably hydrogen;
   (3) R₃ is hydrogen, (C₁₋₈)alkyl or an optionally substituted (C₁₋₈)alkylOC(═O)NH, (C₃₋₈)cycloalkylOC(═O)NH, (C₃₋₈)cycloalkyl(C₁₋₄)alkylOC(═O)NH, aryl(C₁₋₄)alkylOC(═O)NH, heteroaryl-(C₁₋₄)alkylOC(═O)NH, (C₁₋₄)alkylC(═O)NH, (C₃₋₈)cycloalkylC(═O)NH, arylC(═O)NH, aryl(C₁₋₄)-alkylC(═O)NH, heteroarylC(═O)NH or heteroaryl(C₁₋₄)alkylC(═O)NH group;
   preferably hydrogen;
   (4) U is a bond, CF₂, CF₂CF₂, CHF, CHFCHF, cycloprop-1,2-ylene, (C₁₋₃)alkylenoxy, (C₁₋₃)-alkylenamino, (C₁₋₈)alkylene, NRₑ or an aromatic or heteroaromatic ring, which ring is optionally substituted with halogen, (C₁₋₈)alkoxy, hydroxy or (C₁₋₈)alkyl, whereby Z and V are in ortho- or meta-position to each other, wherein
Rₑ is hydrogen, (C₁₋₈)alkyl or (C₃₋₇)cycloalkyl;
   preferably a bond or (C₁₋₃)alkylenoxy;
   (5) V is CH═CH, cycloprop-1,2-ylene, CH₂CH(OH), CH(OH)CH₂ or CR_{f}R_{f}CR_{f}R_{f}, wherein
each R_{f}, independently, is hydrogen, fluorine or (C₁₋₈)alkyl;
   preferably CH₂CH₂;
   (6) either
   V₁ is hydrogen and
   V₂ is hydroxy
   or
   V₁ and V₂ together are oxo;
   preferably V₁ is hydrogen and V₂ is hydroxy;
   (7) W is (C₁₋₈)alkylene, O, S, S(═O)₂, C(═O), C(═O)O, OC(═O), N(R_{g})C(═O), C(═O)NR_{g} or NR_{g}, wherein
R_{g} is hydrogen or (C₁₋₈)alkyl;
   preferably (C₁₋₈)alkylene;
   preferably (C₁₋₄)alkylene;
   preferably CH(CH₃);
   (8) X is an optionally substituted aromatic or heteroaromatic ring, whereby Y and C(═O)NR₂ are in meta-position to each other;
   preferably an optionally substituted phenyl or pyridyl ring, the optional substituents being independently selected from the group, consisting of halogen, (C₁₋₈)alkyl, (C₁₋₆)alkoxy, (C₁₋₄)-alkoxy(C₁₋₄)alkyl, heteroaryl and N,N-di[(C₁₋₄)alkyl]aminocarbonyl;
   preferably a mono-substituted phenyl or pyridyl ring, the substituent being selected from the group, consisting of halogen, (C₁₋₆)alkyl, (C₁₋₆)alkoxy, (C₁₋₄)alkoxy(C₁₋₄)alkyl, oxazolyl and N,N-di[(C₁₋₄)alkyl]aminocarbonyl;
   (9) Y is a bond, O, S(═O)₂, S(═O)₂NRₕ, N(Rₕ)S(═O)₂, NRₕ, C(Rₕ)OH, C(═O)NRₕ, N(Rₕ)C(═O), C(═O)N(Rₕ)O or ON(Rₕ)C(═O), wherein
Rₕ is hydrogen, (C₁₋₈)alkyl or (C₃₋₈)cycloalkyl;
   preferably O or NRₕ, wherein
Rₕ is hydrogen, (C₁₋₈)alkyl or (C₃₋₈)cycloalkyl;
   preferably O or NH;
   (10) Z is O, CH₂, CF₂, CHF, CH═CH, cycloprop-1,2-ylene or a bond;
   preferably CH₂ or CH═CH;
   (11) n is 0 to 5;
   preferably 0 to 3;
   preferably 0 or 3;
   (12) the number of ring atoms included in the macrocyclic ring is 14, 15, 16 or 17; preferably 16.

The preferred embodiments (1) to (12) are preferred independently, collectively or in any combination or sub-combination.

In especially preferred embodiments, the invention relates to one or more than one of the compounds of the formula I mentioned in the Examples hereinafter, in free base form or in acid addition salt form.

In a further aspect, the invention relates to a process for the preparation of a compound of the formula I, in free base form or in acid addition salt form, comprising the steps of

a) for the preparation of a compound of the formula I, in which R₁ is N(Rₐ)R_{b}, V₁ is hydrogen and V₂ is hydroxy, reaction of a compound of the formula

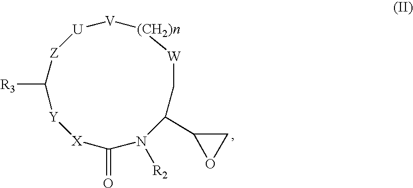

in which R₂, R₃, U, V, W, X, Y, Z and n are as defined for the formula I, with a compound of the formula HN(Rₐ)R_{b} (III), in which Rₐ and R_{b} are as defined for the formula I, or
b) cyclisation by metathesis of a suitable open chain-precursor compound, which carries, in each case, a carbon-carbon double bond at each of the two ends of the said open chain, in the presence of a catalyst, for instance a ruthenium, tungsten or molybdenum complex,
in each case optionally followed by reduction, oxidation or other functionalisation of the resulting compound and/or by cleavage of any protecting group(s) optionally present,
and of recovering the so obtainable compound of the formula I in free base form or in acid addition salt form.

The reactions can be effected according to conventional methods, for example as described in the Examples.

The working-up of the reaction mixtures and the purification of the compounds thus obtainable may be carried out in accordance with known procedures.

Acid addition salts may be prepared from free bases in known manner, and vice-versa.

Compounds of the formula I can also be prepared by further conventional processes, which processes are further aspects of the invention, e.g. as described in the Examples.

The starting materials of the formulae II and III and the open chain-precursor compounds, which are used according to process variant b), are known or may be prepared according to conventional procedures starting from known compounds, for example as described in the Examples.

Compounds of the formula I, in free base form or in pharmaceutically acceptable acid addition salt form, hereinafter often referred to as “agents of the invention”, exhibit valuable pharmacological properties, when tested in vitro or in vivo, and are, therefore, useful in medicaments.

E. g., agents of the invention are inhibitors of aspartic proteases and can be used for the treatment of a condition, disease or disorder involving processing by such enzymes. Particularly, agents of the invention inhibit beta-secretase and, thus, the generation of beta-amyloid and the subsequent aggregation into oligomers and fibrils.

The inhibiting properties of an agent of the invention towards proteases can be evaluated, e.g., in a test as described hereinafter.

### Test 1: Inhibition of Human BACE

Recombinant BACE (extracellular domain, expressed in baculovirus and purified using standard methods) at 0.1 to 10 nM concentrations is incubated with the test compound at various concentrations for 1 hour at room temperature in 10 to 100 mM acetate buffer, pH 4.5, containing 0.1% CHAPS. Synthetic fluorescence-quenched peptide substrate, derived from the sequence of APP and containing a suitable fluorophore-quencher pair, is added to a final concentration of 1 to 5 μM, and the increase in fluorescence is recorded at a suitable excitation/emission wavelength in a microplate spectro-fluorimeter for 5 to 30 minutes in 1-minute intervals. IC₅₀ values are calculated from percentage of inhibition of BACE-activity as a function of the test compound concentration.

### Test 2: Inhibition of Human BACE-2

Recombinant BACE-2 (extracellular domain, expressed in baculovirus and purified using standard methods) at 0.1 to 10 nM concentrations is incubated with the test compound at various concentrations for 1 hour at room temperature in 10 to 100 mM acetate buffer, pH 4.5, containing 0.1% CHAPS. Synthetic peptide substrate, derived from the sequence of APP and containing a suitable fluorophore-quencher pair, is added to a final concentration of 1 to 5 μM, and the increase in fluorescence is recorded at a suitable excitation/emission wavelength in a microplate spectro-fluorimeter for 5 to 30 minutes in 1-minute intervals. IC₅₀ values are calculated from percentage of inhibition of BACE-2-activity as a function of the test compound concentration.

### Test 3: Inhibition of Human Cathepsin D

Recombinant cathepsin D (expressed as procathepsin D in baculovirus, purified using standard methods and activated by incubation in sodium formate buffer pH 3.7) is incubated with the test compound at various concentrations for 1 hour at room temperature in sodium formate or sodium acetate buffer at a suitable pH within the range of pH 3.0 to 5.0. Synthetic peptide substrate Mca-Gly-Lys-Pro-Ile-Leu-Phe-Phe-Arg-Leu-Lys(DNP)-D-Arg-NH₂ is added to a final concentration of 1 to 5 μM, and the increase in fluorescence is recorded at excitation of 325 nm and emission at 400 nm in a microplate spectro-fluorimeter for 5 to 30 minutes in 1-minute intervals. IC₅₀ values are calculated from the percentage of inhibition of cathepsin D-activity as a function of the test compound concentration.

### Test 4: Inhibition of Cellular Release of Amyloid Peptide 1-40

Chinese hamster ovary cells are transfected with the gene for amyloid precursor protein. The cells are plated at a density of 8000 cells/well into 96-well microtiter plates and cultivated for 24 hours in DMEM cell culture medium containing 10% FCS. The test compound is added to the cells at various concentrations, and the cells are cultivated for 24 hours in the presence of the test compound. The supernatants are collected, and the concentration of amyloid peptide 1-40 is determined using sandwich ELISA. The potency of the compound is calculated from the percentage of inhibition of amyloid peptide release as a function of the test compound concentration.

In at least one of the above-described tests, agents of the invention show activity at concentrations below 50 μM.

Specifically, the agent of the invention described in Example 7 shows an IC₅₀ value of 0.04 μM in Test 1.

Due to their inhibiting properties towards proteases, agents of the invention are useful, e.g., in the treatment or prevention of a neurological or vascular condition, disease or disorder, in which beta-amyloid generation or aggregation plays a role, such as a neurodegenerative condition, disease or disorder, e.g. Alzheimer's disease, Down's syndrome, memory impairment, cognitive impairment, dementia, amyloid neuropathies, brain inflammation, nerve trauma, brain trauma, vascular amyloidosis or cerebral haemorrhage with amyloidosis, or, based on the inhibition of BACE-2 (beta-site APP-cleaving enzyme 2) or cathepsin D, which are close homologues of the pepsin-type aspartyl proteases and beta-secretase, and the correlation of the BACE-2 or cathepsin D expression with a more tumorigenic or metastatic potential of tumor cells, in the suppression of the metastasis process associated with tumor cells.

For the above-mentioned indications, the appropriate dosage will vary depending on, e.g., the compound employed as active pharmaceutical ingredient, the host, the mode of administration, the nature and severity of the condition, disease or disorder or the effect desired. However, in general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range of from about 0.5 to about 2000, preferably from about 2 to about 200, mg of an agent of the invention conveniently administered, for example, in divided doses up to four times a day or in sustained release form.

An agent of the invention may be administered by any conventional route, in particular enterally, preferably orally, e.g. in the form of a tablet or capsule, or parenterally, e.g. in the form of an injectable solution or suspension.

In accordance with the foregoing, in a further aspect, the invention relates to an agent of the invention for use as a medicament, e.g. for the treatment or prevention of a neurological or vascular condition, disease or disorder, in which beta-amyloid generation or aggregation plays a role, or for the suppression of the metastasis process associated with tumor cells.

In a further aspect, the invention relates to the use of an agent of the invention as active pharmaceutical ingredient in a medicament, e.g. for the treatment or prevention of a neurological or vascular condition, disease or disorder, in which beta-amyloid generation or aggregation plays a role, or for the suppression of the metastasis process associated with tumor cells.

In a further aspect, the invention relates to a pharmaceutical composition comprising an agent of the invention as active pharmaceutical ingredient in association with at least one pharmaceutically acceptable carrier or diluent. Such a composition may be manufactured in conventional manner, e.g. by mixing its components. Unit dosage forms contain, e.g., from about 0.1 to about 1000, preferably from about 1 to about 500, mg of an agent of the invention.

An agent of the invention can be administered as sole active pharmaceutical ingredient or as a combination with at least one other active pharmaceutical ingredient effective, e.g., in the treatment or prevention of a neurological or vascular condition, disease or disorder, in which beta-amyloid generation or aggregation plays a role, or in the suppression of the metastasis process associated with tumor cells. Such a pharmaceutical combination may be in the form of a unit dosage form, which unit dosage form comprises a predetermined quantity of each of the at least two active components in association with at least one pharmaceutically acceptable carrier or diluent. Alternatively, the pharmaceutical combination may be in the form of a package comprising the at least two active components separately, e.g. a pack or dispenser-device adapted for the concomitant or separate administration of the at least two active components, in which these active components are separately arranged. In a further aspect, the invention relates to such pharmaceutical combinations.

In a further aspect, the invention relates to the use of an agent of the invention for the manufacture of a medicament for the treatment or prevention of a neurological or vascular condition, disease or disorder, in which beta-amyloid generation or aggregation plays a role, or for the suppression of the metastasis process associated with tumor cells.

In a further aspect, the invention relates to a method for the treatment or prevention of a neurological or vascular condition, disease or disorder, in which beta-amyloid generation or aggregation plays a role, or for the suppression of the metastasis process associated with tumor cells, in a subject in need of such treatment, prevention or suppression, which method comprises administering to such subject an effective amount of an agent of the invention.

The following Examples illustrate the invention, but do not limit it.

### EXAMPLES

### Abbreviations

AcCN acetonitrile
AcOH acetic acid
aq. aqueous
b.p. boiling point
BINAP (±)-1,1′-binaphthaline-2,2′-diyl-bis-(diphenylphosphine)
Boc tert-butoxycarbonyl
Cbz-Cl benzyl chloroformate
conc. concentrated
DBU diazabicycloundecene
DCM dichloromethane
DIPEA diisopropylethylamine
DMAP 4-dimethylaminopyridine
DMF dimethylformamide
DMPU N,N′-dimethylpropylene urea
DMSO dimethylsulfoxide
EDC.HCl 1-ethyl-3-[3-(dimethylamino)propyl]-carbodiimide hydrochloride
ES electron spray
Et₂O diethyl ether
EtOAc ethyl acetate
EtOH ethanol
Grubbs II
catalyst 1,3-bis-(2,4,6-trimethylphenyl)-2-imidazolidinylidene)dichloro(phenylmethylene)-(tricyclohexylphosphine)ruthenium
h hour(s)
¹H-NMR proton nuclear magnetic resonance spectrometry
HOBt hydroxybenzotriazole
HPLC high pressure liquid chromatography
LC liquid chromatography
LDA lithium diisopropylamide
m.p. melting point
MeOH methanol
min minute(s)
MS mass spectrometry
NH₃ 13.4 N aq. ammonia
PPTS pyridinium-para-toluenesulfonate
Rf retention factor (thin layer chromatography)
rt room temperature
SK-CC02-A 2-(dimethylamino)ferrocen-1-yl-palladium(II)chloride dinorbornylphosphine complex
TBME tert-butyl methyl ether
TFA trifluoroacetic acid
THF tetrahydrofuran

### Example 1

### (10R,12S)-12-{(R)-2-[1-(4-tert-Butyl-pyrid-2-yl)-cyclopropylamino]-1-hydroxy-ethyl}-17-methoxymethyl-10-methyl-2,13-diaza-bicyclo[13.3.1]nonadeca-1(19),15,17-trien-14-one

### a) (1S,3R)-1-((S)-2-Chloro-1-hydroxy-ethyl)-3-methyl-hept-6-enyl amino hydrochloride

A solution of 709 mg (2.32 mmol) [(1S,3R)-1-((S)-2-chloro-1-hydroxy-ethyl)-3-methyl-hept-6-enyl]-carbamic acid tert-butyl ester in 5 ml DCM is cooled to 0° C. and 7.0 ml 5 M HCl in Et₂O (35 mmol) are added. The mixture is stirred at rt for 1.5 h. The solvent is evaporated to yield the desired product as pale brownish powder (566 mg), which is used for the next step without further purification.

MS (LC/MS): 205.9=[MH]⁺.

### b) {3-[(1S,3R)-1-((S)-2-Chloro-1-hydroxy-ethyl)-3-methyl-hept-6-enylcarbamoyl]-5-methoxymethyl-phenyl}-pent-4-enyl-carbamic acid benzyl ester

To an ice-cold solution of 1.23 g (3.2 mmol) 3-(benzyloxycarbonyl-pent-4-enyl-amino)-5-methoxymethyl-benzoic acid (A2), 693 mg (4.48 mmol) HOBt.H₂O, 0.559 ml (3.2 mmol) DIPEA and 775 mg (3.2 mmol) 1(S)-(2-chloro-1(S)-hydroxy-ethyl)-3(R)-methyl-hept-6-enyl hydrochloride in 16 ml DCM are added 751 mg (3.84 mmol) EDC.HCl. The mixture is stirred at rt for 17 h. After cooling with an ice bath 10.5 ml of 1.0 M HCl are added and the layers are separated. The organic layer is washed with 1 M potassium bicarbonate, water, dried with sodium sulfate and evaporated. The residue is purified by chromatography on silica gel (toluene/EtOH 97/3) and gives the product as a yellow solid.

¹H-NMR (400 MHz, d6-DMSO): 8.15 (d, 1H), 7.66 (d, 2H), 7.34 (s, 1H), 7.32-7.21 (m, 4H), 5.78-5.66 (m, 2H), 5.37 (d, 1H), 5.07 (s, 2H), 4.97-4.80 (m, 4H), 4.43 (s, 2H), 4.11-4.02 (m, 1H), 3.69-3.59 (m, 4H), 3.49-3.42 (m, 1H), 3.29 (s, 3H), 2.04-1.95 (m, 4H), 1.65-1.38 (m, 5H), 1.35-1.17 (m, 3H), 0.83 (d, 3H).

### c) (E/Z)-(10R,12S)-12-((S)-2-Chloro-1-hydroxy-ethyl)-17-methoxymethyl-10-methyl-14-oxo-2,13-diaza-bicyclo[13.3.1]nonadeca-1 (19),6,15,17-tetraene-2-carboxylic acid benzyl ester

A solution of 1.18 g (2.07 mmol) {3-[(1S,3R)-1-((S)-2-chloro-1-hydroxy-ethyl)-3-methyl-hept-6-enylcarbamoyl]-5-methoxymethyl-phenyl}-pent-4-enyl-carbamic acid benzyl ester in 10.4 ml DCM is added dropwise within an hour to a refluxing solution of 88 mg of Grubbs II catalyst in 207 ml of DCM. The mixture is refluxed for additional 30 min, 0.62 ml of butylvinylether are added and stirring is continued for 30 min. The mixture is poured onto a silica gel column and chromatographed (DCM to DCM/MeOH 98/2) to give the product as a brownish foam.

¹H-NMR (400 MHz, d6-DMSO): 8.13 (d, 1H), 7.56 (s, 1H), 7.52 (s, 1H), 7.45 (s, 1H), 7.35-7.27 (m, 5H), 5.49-5.27 (m, 2H), 5.19 (d, 1H), 5.07 (d, 1H), 4.42 (s, 2H), 4.06-3.97 (m, 1H), 3.92-3.81 (m, 1H), 3.70-3.64 (m, 1H), 3.63-3.54 (m, 1H), 3.50-3.44 (m, 1H), 3.28 (s, 3H), 2.12-1.89 (m, 4H), 1.71-1.38 (m, 5H), 1.34-1.19 (m, 3H), 0.74 (d, 3H).

### d) (10R,12S)-12-((S)-2-Chloro-1-hydroxy-ethyl)-17-methoxymethyl-10-methyl-2,13-diaza-bicyclo[13.3.1]nonadeca-1(19),15,17-trien-14-one

A solution of 895 mg (1.65 mmol) of (E/Z)-(10R,12S)-12-((S)-2-chloro-1-hydroxy-ethyl)-17-methoxymethyl-10-methyl-14-oxo-2,13-diaza-bicyclo[13.3.1]nonadeca-1(19),6,15,17-tetraene-2-carboxylic acid benzyl ester in 16.5 ml EtOH is stirred at rt in the presence of 330 mg 10% Pd/C under a hydrogen atmosphere for 4 h. The catalyst is filtered off and the filtrate evaporated. The residue is dissolved in 50 ml EtOH/DCM (90/10) and stirred at rt in the presence of 330 mg 10% Pd/C under a hydrogen atmosphere for 3 h. The catalyst is filtered off and the filtrate evaporated. The residue is purified by chromatography on silica gel (DCM/MeOH 99/1 to 98/2) and gives the title compound as a grey solid.

¹H-NMR (400 MHz, d6-DMSO): 7.99 (d, 1H), 6.80 (s, 1H), 6.79 (s, 1H), 6.62 (s, 1H), 5.97-5.90 (m, 1H), 5.35 (d, 1H), 4.28 (s, 2H), 4.03-3.94 (m, 1H), 3.64-3.59 (m, 1H), 3.58-3.51 (m, 1H), 3.49-3.40 (m, 2H), 3.26 (s, 3H), 2.93-2.82 (m, 1H), 1.71-1.60 (m, 2H), 1.58-1.17 (m, 12H), 1.02-0.93 (m, 1H), 0.83 (d, 3H).

### e) (10R,12S)-17-Methoxymethyl-10-methyl-12-(S)-oxiranyl-2,13-diaza-bicyclo-[13.3.1]nonadeca-1(19),15,17-trien-14-one

To a solution of 323 mg (0.78 mmol) (10R,12S)-12-((S)-2-chloro-1-hydroxy-ethyl)-17-methoxymethyl-10-methyl-2,13-diaza-bicyclo[13.3.1]nonadeca-1(19),15,17-trien-14-one in 1.6 ml THF are added dropwise at 0° C. 1.6 ml aqueous 1 M sodium hydroxide and the reaction mixture is stirred at 0° C. for 2 h. 15.7 ml of a aqueous half-saturated ammonium chloride solution are added and the mixture is extracted with DCM. The combined organic layers are washed with water, dried with sodium sulfate and evaporated to give the product as a colorless solid.

¹H-NMR (400 MHz, d6-DMSO): 8.09 (d, 1H), 6.79 (br s, 2H), 6.62 (s, 1H), 5.99-5.92 (m, 1H), 4.28 (s, 2H), 3.89-3.80 (m, 1H), 3.51-3.40 (m, 1H), 3.25 (s, 3H), 2.94-2.84 (m, 2H), 2.72-2.68 (m, 1H), 2.67-2.62 (m, 1H), 1.89-1.79 (m, 1H), 1.71-1.09 (m, 13H), 1.03-0.92 (m, 1H), 0.83 (d, 3H).

### f) (10R,12S)-12-{(R)-2-[1-(4-tert-Butyl-pyrid-2-yl)-cyclopropylamino]-1-hydroxy-ethyl}-17-methoxymethyl-10-methyl-2,13-diaza-bicyclo[13.3.1]nonadeca-1(19),15,17-trien-14-one

A solution of 79 mg (0.2 mmol) (10R,12S)-17-methoxymethyl-10-methyl-12-(S)-oxiranyl-2,13-diaza-bicyclo[13.3.1]nonadeca-1(19),15,17-trien-14-one and 145 mg (0.76 mmol) 1-(4-tertbutyl-pyrid-2-yl)-cyclopropylamine in 0.66 ml DCM and 0.1 ml DMF is warmed to 80° C. After the DCM is evaporated stirring is continued for 8 h. The reaction mixture is dissolved in MeOH and purified by preparative HPLC (Xterra RP18, 19×150 mm, 5 μm, 10-100% AcCN (20 min), 25 ml/min). The crude product is then purified by preparative thin layer chromatography on silica gel (DCM/MeOH 90/10) yielding a colorless solid.

¹H-NMR (400 MHz, d6-DMSO): 8.27 (d, 1H), 7.91 (d, 1H), 7.66 (s, 1H), 7.06 (dd, 1H), 6.79 (s, 1H), 6.75 (s, 1H), 6.60 (s, 1H), 5.93-5.87 (m, 1H), 4.75 (d, 1H), 4.26 (s, 2H), 3.99-3.90 (m, 1H), 3.52-3.40 (m, 2H), 3.25 (s, 3H), 2.91-2.81 (m, 1H), 2.68-2.52 (m, 2H), 1.71-1.60 (m, 2H), 1.58-1.11 (m, 15H), 1.22 (s, 9H), 1.01-0.90 (m, 3H), 0.82 (d, 3H).

### Example 1a

### (10R,12S)-12-((R)-2-{1-[5-(2,2-Dimethyl-propyl)-isoxazol-3-yl]-cyclopropylamino}-1-hydroxy-ethyl)-17-methoxymethyl-10-methyl-2,13-diaza-bicyclo-[13.3.1]nonadeca-1(19),15,17-trien-14-one

The title compound is prepared similarly to example 1, using 1-[5-(2,2-dimethyl-propyl)-isoxazol-3-yl]-cyclopropylamine (building block C5) instead of 1-(4-tert-butyl-pyrid-2-yl)-cyclopropylamine (building block C1) in step f).

¹H-NMR (400 MHz, d6-DMSO): 7.92 (d, 1H), 6.80 (br s, 2H), 6.61 (s, 1H), 6.14 (s, 1H), 5.91 (dd, 1H), 4.51 (d, 1H), 4.28 (s, 2H), 3.97-3.88 (m, 1H), 3.51-3.41 (m, 1H), 3.38-3.33 (m, 1H), 3.27 (s, 3H), 2.91-2.81 (m, 1H), 2.77-2.69 (m, 1H), 2.66-2.55 (m, 2H), 2.54 (s, 2H), 1.68-1.59 (m, 2H), 1.56-1.15 (m, 12H), 1.07-0.89 (m, 5H), 0.87 (s, 9H), 0.81 (d, 3H).

### Example 2

### (10R,12S)-12-{(R)-2-[1-(4-tert-Butyl-pyrid-2-yl)-cyclopropylamino]-1-hydroxy-ethyl}-10-methyl-17-oxazol-2-yl-2,13-diaza-bicyclo[13.3.1]nonadeca-1(19),15,17-trien-14-one

The title compound is prepared similarly to example 1, using 3-(benzyloxycarbonyl-pent-4-enyl-amino)-5-oxazol-2-yl-benzoic acid (building block A3) instead of 3-(benzyloxycarbonyl-pent-4-enyl-amino)-5-methoxymethyl-benzoic acid (building block A2) in step b).

¹H-NMR (400 MHz, d6-DMSO): 8.26 (d, 1H), 8.16 (s, 1H), 8.08 (d, 1H), 7.65 (s, 1H), 7.40 (s, 1H), 7.33 (s, 1H), 7.30 (s, 1H), 7.04 (dd, 1H), 6.99 (s, 1H), 6.32-6.25 (m, 1H), 4.79 (d, 1H), 4.02-3.92 (m, 1H), 3.58-3.42 (m, 2H), 2.97-2.86 (m, 1H), 2.70-2.53 (m, 2H), 1.75-1.61 (m, 2H), 1.59-1.11 (m, 15H), 1.18 (s, 9H), 1.04-0.91 (m, 3H), 0.84 (d, 3H).

### Example 3

### (10R,12S)-12-{(R)-2-[1-(4-tert-Butyl-pyrid-2-yl)-cyclopropylamino]-1-hydroxy-ethyl}-10-methyl-17-oxazol-2-yl-2-oxa-13-aza-bicyclo[13.3.1]nonadeca-1(19),15,17-trien-14-one

The title compound is prepared similarly to example 1, using 3-oxazol-2-yl-5-pent-4-enyloxy-benzoic acid (building block A4) instead of 3-(benzyloxycarbonyl-pent-4-enyl-amino)-5-methoxymethyl-benzoic acid (building block A2) in step b).

¹H-NMR (400 MHz, d6-DMSO): 8.26 (d, 1H), 8.24 (s, 1H), 8.22 (s, 1H), 7.83 (s, 1H), 7.65 (s, 1H), 7.48 (s, 1H), 7.39 (s, 1H), 7.04 (dd, 1H), 4.87 (d, 1H), 4.64-4.54 (m, 1H), 4.14-3.93 (m, 3H), 3.52-3.45 (m, 1H), 2.71-2.53 (m, 2H), 1.85-1.65 (m, 2H), 1.60-1.11 (m, 15H), 1.17 (s, 9H), 1.04-0.92 (m, 3H), 0.84 (d, 3H).

### Example 4

### (10R,12S)-12-{(R)-2-[1-(3-tert-Butyl-phenyl)-cyclopropylamino]-1-hydroxy-ethyl}-10-methyl-14-oxo-2,13-diaza-bicyclo[13.3.1]nonadeca-1(18),15(19),16-triene-17-carboxylic acid dimethylamide

The title compound is prepared similarly to example 1, using 5-(benzyloxycarbonyl-pent-4-enyl-amino)-N,N-dimethyl-isophthalmic acid (building block A5) instead of 3-(benzyloxycarbonyl-pent-4-enyl-amino)-5-methoxymethyl-benzoic acid (building block A2) in step b) and 1-(3-tert-butyl-phenyl)-cyclopropylamine (building block C3) instead of 1-(4-tert-butyl-pyridin-2-yl)-cyclopropylamine (building block C1) in step f).

¹H-NMR (400 MHz, d6-DMSO): 7.98 (d, 1H), 7.29 (s, 1H), 7.15-7.11 (m, 2H), 7.01-6.90 (m, 1H), 6.88 (s, 1H), 6.74 (s, 1H), 6.62 (s, 1H), 6.12-6.06 (m, 1H), 4.63-4.55 (m, 1H), 3.96-3.87 (m, 1H), 3.55-3.34 (m, 3H), 2.95 (br s, 3H), 2.89 (br s, 3H), 2.48-2.43 (m, 1H), 1.68-1.18 (m, 16H), 1.24 (s, 9H), 1.01-0.83 (m, 4H), 0.81 (d, 3H).

### Example 4a

### (10R,12S)-12-{(R)-2-[1-(4-tert-Butyl-pyrid-2-yl)-cyclopropylamino]-1-hydroxy-ethyl}-10-methyl-14-oxo-2,13-diaza-bicyclo[13.3.1]nonadeca-1(18),15(19),16-triene-17-carboxylic acid dimethylamide

The title compound is prepared similarly to example 1, using 5-(benzyloxycarbonyl-pent-4-enyl-amino)-N,N-dimethyl-isophthalmic acid (building block A5) instead of 3-(benzyloxycarbonyl-pent-4-enyl-amino)-5-methoxymethyl-benzoic acid (building block A2) in step b).

¹H-NMR (400 MHz, d6-DMSO): 8.26 (d, 1H), 8.02 (d, 1H), 7.67 (s, 1H), 7.05 (dd, 1H), 6.91 (s, 1H), 6.74 (s, 1H), 6.62 (s, 1H), 6.11-6.06 (m, 1H), 4.83-4.75 (m, 1H), 4.00-3.91 (m, 1H), 3.54-3.41 (m, 3H), 2.94 (br s, 3H), 2.88 (br s, 3H), 2.66-2.61 (m, 1H), 2.58-2.52 (m, 1H), 1.72-1.11 (m, 16H), 1.22 (s, 9H), 1.02-0.89 (m, 4H), 0.82 (d, 3H).

### Example 5

### (10R,12S)-12-{(R)-2-[1-(4-tert-Butyl-pyrid-2-yl)-cyclopropylamino]-1-hydroxy-ethyl}-10-methyl-14-oxo-2-oxa-13-aza-bicyclo[13.3.1]nonadeca-1(18),15(19),16-triene-17-carboxylic acid dimethylamide

The title compound is prepared similarly to example 1, using N,N-dimethyl-5-pent-4-enyloxy-isophthalmic acid (building block A6) instead of 3-(benzyloxycarbonyl-pent-4-enyl-amino)-5-methoxymethyl-benzoic acid (building block A2) in step b).

¹H-NMR (400 MHz, d6-DMSO): 8.25 (d, 1H), 8.16 (d, 1H), 7.65 (s, 1H), 7.39 (s, 1H), 7.17 (s, 1H), 7.04 (dd, 1H), 6.93 (s, 1H), 4.84 (d, 1H), 4.59-4.49 (m, 1H), 4.08-3.91 (m, 2H), 3.50-3.43 (m, 1H), 2.95 (s, 3H), 2.85 (s, 3H), 2.67-2.51 (m, 2H), 1.82-1.63 (m, 2H), 1.57-1.11 (m, 15H), 1.20 (s, 9H), 1.02-0.90 (m, 3H), 0.82 (d, 3H).

### Example 5a

### (10R,12S)-12-{(R)-2-[1-(5-Bromo-pyrid-3-yl)-cyclopropylamino]-1-hydroxy-ethyl}-10-methyl-14-oxo-2-oxa-13-aza-bicyclo[13.3.1]nonadeca-1(18),15(19),16-triene-17-carboxylic acid dimethylamide

The title compound is prepared similarly to example 1, using N,N-dimethyl-5-pent-4-enyloxy-isophthalmic acid (building block A6) instead of 3-(benzyloxycarbonyl-pent-4-enyl-amino)-5-methoxymethyl-benzoic acid (building block A2) in step b) and 1-(5-bromo-pyrid-3-yl)-cyclopropylamine (building block C4) instead of 1-(4-tert-butyl-pyrid-2-yl)-cyclopropylamine (building block C1) in step f).

¹H-NMR (400 MHz, d6-DMSO): 8.46-8.43 (m, 2H), 8.17 (d, 1H), 7.91-7.89 (m, 1H), 7.36 (s, 1H), 7.19 (s, 1H), 6.95-6.93 (m, 1H), 4.71 (d, 1H), 4.57-4.49 (m, 1H), 4.09-3.91 (m, 2H), 3.43-3.37 (m, 1H), 2.97 (s, 3H), 2.88 (s, 3H), 2.71-2.66 (m, 1H), 1.82-1.73 (m, 2H), 1.69-1.18 (m, 15H), 1.07-0.93 (m, 4H), 0.83 (d, 3H).

### Example 6

### (10R,12S)-17-Chloro-12-{(R)-1-hydroxy-2-[1-(4-isopropyl-pyrid-2-yl)-cyclopropylamino]-ethyl}-10-methyl-2-oxa-13,18-diaza-bicyclo[13.3.1]nonadeca-1(18),15(19),16-trien-14-one

The title compound is prepared similarly to example 1, using 2-chloro-6-pent-4-enyloxy-isonicotinic acid (building block A7) instead of 3-(benzyloxycarbonyl-pent-4-enyl-amino)-5-methoxymethyl-benzoic acid (building block A2) in step b) and 1-(4-isopropyl-pyrid-2-yl)-cyclopropylamine (building block C2) instead of 1-(4-tert-butyl-pyrid-2-yl)-cyclopropylamine (building block C1) in step f).

¹H-NMR (400 MHz, d6-DMSO): 8.46 (d, 1H), 8.26 (d, 1H), 7.47 (s, 1H), 7.18 (s, 1H), 7.17 (s, 1H), 6.96 (dd, 1H), 4.88 (d, 1H), 4.57-4.49 (m, 1H), 4.26-4.16 (m, 1H), 4.01-3.93 (m, 1H), 3.51-3.44 (m, 1H), 2.84-2.76 (m, 1H), 2.65-2.52 (m, 2H), 1.77-1.11 (m, 16H), 1.14 (d, 6H), 1.02-0.93 (m, 4H), 0.83 (d, 3H).

### Example 7

### (10R,12S)-12-{(R)-2-[1-(4-tert-Butyl-pyrid-2-yl)-cyclopropylamino]-1-hydroxy-ethyl}-17-methoxy-10-methyl-2,13-diaza-bicyclo[13.3.1]nonadeca-1(19),15,17-trien-14-one

The title compound can be prepared similarly to example 1, using 3-methoxy-5-pent-4-enylamino-benzoic acid (building block A8) instead of 3-(benzyloxycarbonyl-pent-4-enyl-amino)-5-methoxymethyl-benzoic acid (building block A2) in step b).

¹H-NMR (400 MHz, d6-DMSO): 8.27 (d, 1H), 7.89 (d, 1H), 7.66 (s, 1H), 7.06 (dd, 1H), 6.52 (s, 1H), 6.38 (s, 1H), 5.89-5.82 (m, 1H), 4.79-4.74 (m, 1H), 3.98-3.89 (m, 1H), 3.65 (s, 3H), 3.51-3.40 (m, 2H), 2.90-2.77 (m, 1H), 2.68-2.53 (m, 2H), 1.71-1.59 (m, 2H), 1.56-1.11 (m, 15H), 1.23 (s, 9H), 1.02-0.89 (m, 3H), 0.82 (d, 3H).

### Example 8

### (10S,12S)-12-{(R)-2-[1-(3-tert-Butyl-phenyl)-cyclopropylamino]-1-hydroxy-ethyl}-17-methoxymethyl-10-methyl-7-oxa-2,13-diaza-bicyclo[13.3.1]nonadeca-1(18),15(19),16-trien-14-one

### a) ((1S,3S)-5-Allyloxy-3-methyl-1-(S)-oxiranyl-pentyl)-carbamic acid tert-butyl ester

To an ice-cold solution of 3.71 g (11 mmol) [(1S,3S)-5-allyloxy-1-((S)-2-chloro-1-hydroxy-ethyl)-3-methyl-pentyl]-carbamic acid tert-butyl ester (building block B1) in 22 ml THF are added dropwise 22 ml aqueous 1 M sodium hydroxide (22 mmol), the solution turns turbid. After addition of 11 ml MeOH the clear reaction mixture is stirred at 0° C. for 2.5 h. The mixture is diluted with 220 ml half-saturated aqueous ammonium-chloride solution, the organic solvents are evaporated and the residual solution is extracted with DCM. The combined organic layers are washed with water, dried with sodium sulfate and evaporated. The product is obtained as brownish oil, which is used for the next step without further purification.

¹H-NMR (400 MHz, d6-DMSO, 100° C.): 6.27 (d, 1H), 5.92-5.82 (m, 1H), 5.24-5.07 (m, 2H), 3.90 (d, 2H), 3.42 (t, 2H), 3.34-3.27 (m, 1H), 2.81-2.78 (m, 1H), 2.64-2.61 (m, 1H), 2.57-2.55 (m, 1H), 1.71-1.61 (m, 1H), 1.59-1.51 (m, 2H), 1.44-1.26 (m, 2H), 1.39 (s, 9H), 0.86 (d, 3H).

### b) ((1S,3S)-5-Allyloxy-1-{(R)-2-[1-(3-tert-butyl-phenyl)-cyclopropylamino]-1-hydroxy-ethyl}-3-methyl-pentyl)-carbamic acid tert-butyl ester

To a solution of 1.65 g (5.5 mmol) ((1S,3S)-5-allyloxy-3-methyl-1-(S)-oxiranyl-pentyl)-carbamic acid tert-butyl ester in 27.5 ml EtOH are added 1.46 g (7.72 mmol) 1-(3-tert-butyl-phenyl)-cyclopropylamine (building block C3) and the mixture is heated to 50° C. for 44 h. The solvent is evaporated and the residue is purified by two successive chromatographies on silica gel (cyclohexane/EtOAc 60/40) and gives the title compound as pale brownish oil.

¹H-NMR (400 MHz, d6-DMSO): 7.32 (br s, 1H), 7.17-7.15 (m, 2H), 7.02-6.99 (m, 1H), 6.41 (d, 1H), 5.90-5.80 (m, 1H), 5.23-5.08 (m, 2H), 4.48 (d, 1H), 3.88 (d, 2H), 3.39-3.33 (m, 2H), 3.27-3.21 (m, 2H), 2.40-2.32 (m, 2H), 1.56-1.23 (m, 6H), 1.33 (s, 9H), 1.28 (s, 9H), 0.92-0.81 (m, 4H), 0.79 (d, 3H).

### c) ((2R,3S,5S)-7-Allyloxy-3-tert-butoxycarbonylamino-2-hydroxy-5-methyl-heptyl)-[1-(3-tert-butyl-phenyl)-cyclopropyl]-carbamic acid benzyl ester

To a solution of 886 mg (1.81 mmol) ((1S,3S)-5-allyloxy-1-{(R)-2-[1-(3-tert-butyl-phenyl)-cyclopropylamino]-1-hydroxy-ethyl}-3-methyl-pentyl)-carbamic acid tert-butyl ester in 14.5 ml DCM are added 0.295 ml (1.99 mmol) benzyl chloroformate and the mixture is stirred for 2 h. Then every 30 min 0.054 ml (0.38 mmol) benzyl chloroformate are added (3 times). 30 min after the last addition the reaction mixture is cooled to 0° C. and 22 ml 2 M aqueous ammonia solution are added, the layers are separated and the aqueous phase extracted with DCM. The combined organic layers are washed with water, dried with sodium sulfate and evaporated. The residue is purified by chromatography on silica gel (cyclohexane/EtOAc 90/10 to 80/20) and gives the title compound as colorless oil.

¹H-NMR (400 MHz, d6-DMSO, 121° C.): 7.30-7.19 (m, 5H), 7.17-7.11 (m, 3H), 6.87-6.83 (m, 1H), 5.91-5.82 (m, 2H), 5.24-5.03 (m, 3H), 4.29 (d, 1H), 3.89 (d, 2H), 3.74-3.67 (m, 1H), 3.63-3.57 (m, 1H), 3.44-3.39 (m, 3H), 3.22-3.15 (m, 2H), 1.77-1.69 (m, 1H), 1.65-1.13 (m, 7H), 1.37 (s, 9H), 1.24 (s, 9H), 1.11-1.03 (m, 1H), 0.86 (d, 3H).

### d) ((2R,3S,5S)-7-Allyloxy-3-amino-2-hydroxy-5-methyl-heptyl)-[1-(3-tert-butyl-phenyl)-cyclopropyl]-carbamic acid benzyl ester hydrochloride

To an ice-cold solution of 2.05 g (3.29 mmol) ((2R,3S,5S)-7-allyloxy-3-tert-butoxycarbonyl-amino-2-hydroxy-5-methyl-heptyl)-[1-(3-tert-butyl-phenyl)-cyclopropyl]-carbamic acid benzyl ester in 25 ml DCM are added 4.75 ml (33.7 mmol) 7.1 M HCl in Et₂O and the mixture is stirred for 4 h while it is allowed to warm to rt. The solvent is evaporated to give the title compound as yellowish foam, which is used for the next step without further purification.

¹H-NMR (400 MHz, d6-DMSO, 121° C.): 7.69 (br s, 3H), 7.29-7.11 (m, 8H), 6.91-6.88 (m, 1H), 5.93-5.82 (m, 1H), 5.25-5.02 (m, 4H), 4.10-4.05 (m, 1H), 3.91 (d, 2H), 3.61 (d, 1H), 3.43 (t, 2H), 3.29-3.24 (m, 1H), 3.18-3.14 (m, 1H), 1.79-1.67 (m, 2H), 1.64-1.20 (m, 6H), 1.24 (s, 9H), 1.12-1.06 (m, 1H), 0.87 (d, 3H).

### e) Allyl-{3-[1S,3S)-5-allyloxy-1-((R)-2-{benzyloxycarbonyl-[1-(3-tert-butyl-phenyl)-cyclopropyl]-amino}-1-hydroxy-ethyl)-3-methyl-pentylcarbamoyl]-5-methoxymethyl-phenyl}-carbamic acid benzyl ester

To an ice-cold solution of 587 mg (1.05 mmol) ((2R,3S,5S)-7-allyloxy-3-amino-2-hydroxy-5-methyl-heptyl)-[1-(3-tert-butyl-phenyl)-cyclopropyl]-carbamic acid benzyl ester hydrochloride, 410 mg (1.15 mmol) 3-(benzyloxycarbonyl-pent-4-enyl-amino)-5-methoxymethyl-benzoic acid (A2) and 227 mg (1.47 mmol) HOBt.H₂O in 6 ml DCM are added 0.183 ml (1.05 mmol) DIPEA and 246 mg (1.26 mmol) EDC.HCl, the mixture is stirred at rt for 17 h. The reaction mixture is diluted with 1 ml EtOH and washed with 1 M aqueous potassium hydrogencarbonate, 0.5 M aqueous HCl and half-saturated aqueous sodium chloride solution. The organic layer is dried with sodium sulfate, evaporated and the residue is purified by chromatography on silica gel (cyclohexane/EtOAc 95/5 to 55/45) and gives the product as yellowish resin.

¹H-NMR (400 MHz, d6-DMSO, 121° C.): 7.67 (d, 1H), 7.61 (d, 2H), 7.31-7.18 (m, 11H), 7.14-7.09 (m, 3H), 6.84 (m, 1H), 5.91-5.77 (m, 2H), 5.18-5.02 (m, 8H), 4.47 (d, 1H), 4.42 (s, 2H), 4.29 (d, 2H), 4.07-3.99 (m, 1H), 3.89-3.84 (m, 3H), 3.68-3.63 (m, 1H), 3.41 (t, 2H), 3.31 (s, 3H), 3.28-3.22 (m, 1H), 1.77-1.72 (m, 1H), 1.69-1.35 (m, 6H), 1.29-1.17 (m, 1H), 1.20 (s, 9H), 1.09-1.03 (m, 1H), 0.88 (d, 3H).

### f) (E/Z)-(10S,12S)-12-((R)-2-{Benzyloxycarbonyl-[1-(3-tert-butyl-phenyl)-cyclopropyl]-amino}-1-hydroxy-ethyl)-17-methoxymethyl-10-methyl-14-oxo-7-oxa-2,13-diaza-bicyclo[13.3.1]nonadeca-1(19),4,15,17-tetraene-2-carboxylic acid benzyl ester

A solution of 774 mg (1.0 mmol) allyl-{3-[1S,3S)-5-allyloxy-1-((R)-2-{benzyloxycarbonyl-[1-(3-tert-butyl-phenyl)-cyclopropyl]-amino}-1-hydroxy-ethyl)-3-methyl-pentylcarbamoyl]-5-methoxymethyl-phenyl}-carbamic acid benzyl ester in 10 ml DCM is added dropwise within 30 min to a refluxing solution of 42 mg [1,3-bis-(2,4,6-trimethylphenyl)-2-imidazolidinylidene)-dichloro(phenylmethylene)-(tricyclohexylphosphine)ruthenium] (Grubbs II catalyst) in 80 ml DCM. Reaction control by TLC and LC-MS shows that no starting material is left, 0.6 ml butylvinylether are added and stirring is continued for 30 min. The reaction mixture is evaporated to a volume of 10 ml, poured onto a silica gel column and chromatographed (cyclohexane/EtOAc 80/20 to 40/60) to give the product as a colorless foam.

MS (ES+): 832.5=[M+H]⁺

### g) (10S,12S)-12-{(R)-2-[1-(3-tert-Butyl-phenyl)-cyclopropylamino]-1-hydroxy-ethyl}-17-methoxymethyl-10-methyl-7-oxa-2,13-diaza-bicyclo[13.3.1]nonadeca-1(18),15(19),16-trien-14-one

To a solution of 458 mg (0.55 mmol) (E/Z)-(10S,12S)-12-((R)-2-{benzyloxycarbonyl-[1-(3-tert-butyl-phenyl)-cyclopropyl]-amino}-1-hydroxy-ethyl)-17-methoxymethyl-10-methyl-14-oxo-7-oxa-2,13-diaza-bicyclo[13.3.1]nonadeca-1(19),4,15,17-tetraene-2-carboxylic acid benzyl ester in 10 ml MeOH are added 0.5 ml 13.4 N aqueous ammonia and 300 mg Raney-Ni, the reaction mixture is stirred under a hydrogen atmosphere for 23 h. The catalyst is removed by filtration, the organic solvent is evaporated the aqueous phase basified with 13.4 N aqueous ammonia and extracted with DCM. The combined organic layers are dried with sodium sulfate and evaporated. Due to incomplete reaction the residue is dissolved in 100 ml MeOH, 15 ml 13.4 N aqueous ammonia and 500 mg Raney-Ni are added and the mixture stirred under a hydrogen atmosphere for 1.75 h. After work-up as described for the first hydrogen-nation the residue is purified by chromatography on silica gel (EtOAc to EtOAc/EtOH 95/5) and gives the product as colorless foam.

¹H-NMR (400 MHz, d6-DMSO): 7.81 (d, 1H), 7.29 (s, 1H), 7.19-7.11 (m, 2H), 7.01 (d, 1H), 6.73 (d, 2H), 6.58 (s, 1H), 5.95 (t, 1H), 4.54 (d, 1H), 4.27 (s, 2H), 3.95-3.87 (m, 1H), 3.59-3.24 (m, 9H), 3.26 (s, 3H), 2.94-2.84 (m, 1H), 1.93-1.72 (m, 2H), 1.71-1.59 (m, 2H), 1.49-1.21 (m, 5H), 1.25 (s, 9H), 0.98-0.87 (m, 3H), 0.85-0.78 (m, 1H), 0.80 (d, 3H).

### Example 9

### (10S,12S)-12-{(R)-2-[1-(4-tert-Butyl-pyrid-2-yl)-cyclopropylamino]-1-hydroxy-ethyl}-17-methoxymethyl-10-methyl-7-oxa-2,13-diaza-bicyclo[13.3.1]nonadeca-1(18),15(19),16-trien-14-one

The title compound is prepared similarly to example 8, using 1-(4-tert-butyl-pyrid-2-yl)-cyclopropylamine (building block C1) instead of 1-(3-tert-butyl-phenyl)-cyclopropylamine (building block C3) in step b) and hydrogenation of the double bond with Raney-Ni in EtOH followed by removal of Cbz with 10% Pd—C in EtOH in step g).

¹H-NMR (400 MHz, d6-DMSO): 8.27 (d, 1H), 7.85 (d, 1H), 7.67 (br s, 1H), 7.06 (dd, 1H), 6.76 (s, 1H), 6.71 (s, 1H), 6.58 (s, 1H), 5.95 (t, 1H), 4.73 (d, 1H), 4.26 (s, 2H), 3.98-3.90 (m, 1H), 3.60-3.50 (m, 2H), 3.47-3.28 (m, 4H), 3.25 (s, 3H), 2.94-2.85 (m, 1H), 2.69-2.55 (m, 2H), 1.93-1.60 (m, 4H), 1.50-1.12 (m, 8H), 1.23 (s, 9H), 1.00-0.89 (m, 2H), 0.81 (d, 3H).

### Example 10

### (10S,12S)-12-{(R)-2-[1-(3-tert-Butyl-phenyl)-cyclopropylamino]-1-hydroxy-ethyl}-17-methoxymethyl-10-methyl-7-oxa-2,13,18-triaza-bicyclo[13.3.1]nonadeca-1(18),15(19),16-trien-14-one

The title compound is prepared similarly to example 8, using 2-allylamino-6-methoxymethyl-isonicotinic acid (building block A10) instead of 3-(benzyloxycarbonyl-pent-4-enyl-amino)-5-methoxymethyl-benzoic acid (building block A2) in step e) and hydrogenation of the double bond with Raney-Ni in EtOH followed by removal of Cbz with 10% Pd—C in EtOH in step g).

¹H-NMR (400 MHz, d6-DMSO, 120° C.): 7.57 (d, 1H), 7.36-7.33 (m, 1H), 7.17-7.14 (m, 2H), 7.07-7.03 (m, 1H), 6.70 (s, 1H), 6.53 (s, 1H), 6.19-6.15 (m, 1H), 4.27 (s, 2H), 4.07-4.03 (m, 1H), 3.97-3.90 (m, 1H), 3.57-3.50 (m, 2H), 3.47-3.33 (m, 3H), 3.36 (s, 3H), 3.11-3.02 (m, 1H), 2.63-2.52 (m, 2H), 1.85-1.71 (m, 2H), 1.68-1.26 (m, 8H), 1.29 (s, 9H), 0.99-0.86 (m, 4H), 0.84 (d, 3H).

### Example 11

### (10S,12S)-12-{(R)-2-[1-(3-tert-Butyl-phenyl)-cyclopropylamino]-1-hydroxy-ethyl}-10,17-dimethyl-7-oxa-2,13,18-triaza-bicyclo[13.3.1]nonadeca-1(18),15(19),16-trien-14-one

### a) (10S,12S)-2-Acetyl-12-{(R)-2-[1-(3-tert-butyl-phenyl)-cyclopropylamino]-1-hydroxy-ethyl}-10,17-dimethyl-7-oxa-2,13,18-triaza-bicyclo[13.3.1]nonadeca-1(18),15(19),16-trien-14-one

The title compound is prepared similarly to example 8, using 2-(acetyl-allyl-amino)-6-methyl-isonicotinic acid (building block A9) instead of 3-(benzyloxycarbonyl-pent-4-enyl-amino)-5-methoxymethyl-benzoic acid (building block A2) in step e), hydrogenation of the double bond and removal of Cbz with Raney-Ni in EtOH in step g).

¹H-NMR (400 MHz, d6-DMSO): 8.26 (d, 1H), 7.38 (s, 1H), 7.35-7.31 (m, 2H), 7.18-7.12 (m, 2H), 7.00-6.97 (m, 1H), 4.70 (d, 1H), 3.97-3.85 (m, 2H), 3.50-3.37 (m, 4H), 3.30-3.24 (m, 2H), 2.60-2.54 (m, 1H), 2.48-2.41 (m, 1H), 1.98 (s, 3H), 1.71-1.63 (m, 2H), 1.61-1.29 (m, 8H), 1.23 (s, 9H), 0.96-0.79 (m, 4H), 0.77 (d, 3H).

### b) (10S,12S)-12-{(R)-2-[1-(3-tert-Butyl-phenyl)-cyclopropylamino]-1-hydroxy-ethyl}-10,17-dimethyl-7-oxa-2,13,18-triaza-bicyclo[13.3.1]nonadeca-1(18),15(19),16-trien-14-one

To a solution of 69 mg (0.12 mmol) (10S,12S)-2-acetyl-12-{(R)-2-[1-(3-tert-butyl-phenyl)-cyclopropylamino]-1-hydroxy-ethyl}-10,17-dimethyl-7-oxa-2,13,18-triaza-bicyclo[13.3.1]-nonadeca-1(18),15(19),16-trien-14-one in 5 ml EtOH are added 0.6 ml 2 M aqueous sodium hydroxide, the mixture is stirred and heated to 60° C. for 2 h. The reaction mixture is diluted with 30 ml water and extracted with DCM, the organic layers are dried with sodium sulfate and evaporated. The residue is purified by preparative thin layer chromatography on silica gel (DCM/MeOH/NH3 90/9/1) to give a colorless resin.

¹H-NMR (400 MHz, d6-DMSO, 120° C.): 7.49 (d, 1H), 7.36-7.33 (m, 1H), 7.17-7.13 (m, 2H), 7.07-7.03 (m, 1H), 6.49 (s, 1H), 6.42 (s, 1H), 6.07-6.02 (m, 1H), 4.07-4.02 (m, 1H), 3.96-3.87 (m, 1H), 3.56-3.29 (m, 5H), 3.10-2.99 (m, 1H), 2.62-2.52 (m, 2H), 2.26 (s, 3H), 1.85-1.70 (m, 2H), 1.68-1.23 (m, 8H), 1.29 (s, 9H), 0.99-0.85 (m, 4H), 0.83 (d, 3H).

### Example 12

### (10S,12S)-12-{(R)-2-[1-(4-tert-Butyl-pyrid-2-yl)-cyclopropylamino]-1-hydroxy-ethyl}-10,17-dimethyl-7-oxa-2,13,18-triaza-bicyclo[13.3.1]nonadeca-1(19),15,17-trien-14-one

The title compound is prepared similarly to example 11, using 1-(4-tert-butyl-pyrid-2-yl)-cyclopropylamine (building block C1) instead of 1-(3-tert-butyl-phenyl)-cyclopropylamine (building block C3) in step b) and hydrogenation of the double bond and removal of Cbz with Raney-Ni in EtOH in step g) as in the synthesis of example 8).

¹H-NMR (400 MHz, d6-DMSO): 8.28 (d, 1H), 8.05 (d, 1H), 7.65 (d, 1H), 7.07 (dd, 1H) 6.62-6.57 (m, 1H), 6.47 (s, 1H), 6.45 (s, 1H), 4.78 (d, 1H), 3.95-3.88 (m, 1H), 3.60-3.50 (m, 2H), 3.46-3.34 (m, 3H), 2.99-2.88 (m, 1H), 2.69-2.52 (m, 3H), 2.23 (s, 3H), 1.84-1.58 (m, 4H), 1.49-1.13 (m, 7H), 1.23 (s, 9H), 1.01-0.92 (m, 2H), 0.81 (d, 3H).

### Example 13

### (E/Z)-(10S,12S)-12-{(R)-2-[1-(3-tert-Butyl-phenyl)-cyclopropylamino]-1-hydroxy-ethyl}-17-methoxymethyl-10-methyl-7-oxa-2,13-diaza-bicyclo[13.3.1]nonadeca-1(18),4,15(19),16-tetraen-14-one

To a solution of 250 mg (0.3 mmol) (E/Z)-(10S,12S)-12-((R)-2-{benzyloxycarbonyl-[1-(3-tert-butyl-phenyl)-cyclopropyl]-amino}-1-hydroxy-ethyl)-17-methoxymethyl-10-methyl-14-oxo-7-oxa-2,13-diaza-bicyclo[13.3.1]nonadeca-1(19),4,15,17-tetraene-2-carboxylic acid benzyl ester in 5 ml DCM are added 0.88 ml (6.0 mmol) iodotrimethylsilane and the reaction mixture is stirred for 10 min, after additional 30 min 3 ml MeOH are added. After addition of 13.4 N aqueous ammonia and water the mixture is extracted with DCM, the combined organic layers are dried with sodium sulfate and evaporated. The residue is dissolved in MeOH and purified by preparative HPLC (Xterra RP18, 19×150 mm, 5 μm, 10-100% AcCN (20 min), 25 ml/min) to give a colorless solid.

¹H-NMR (400 MHz, d6-DMSO): 7.70 (d, 1H), 7.26 (s, 1H), 7.15-7.11 (m, 2H), 7.02-6.99 (m, 1H), 6.89 (s, 1H), 6.80 (s, 1H), 6.67 (s, 1H), 6.16 (t, 1H), 6.05-5.97 (m, 1H), 5.49-5.41 (m, 1H), 4.62 (d, 1H), 4.29 (s, 2H), 3.95-3.79 (m, 3H), 3.78-3.69 (m, 2H), 3.53-3.38 (m, 2H), 3.26 (s, 3H), 2.48-2.42 (m, 2H), 1.58-1.20 (m, 5H), 1.23 (s, 9H), 0.94-0.77 (m, 4H), 0.82 (d, 3H).

### Example 13a

### (E/Z)-(10S,12S)-12-{(R)-2-[1-(4-tert-Butyl-pyrid-2-yl)-cyclopropylamino]-1-hydroxy-ethyl}-17-methoxymethyl-10-methyl-7-oxa-2,13-diaza-bicyclo[13.3.1]nona-deca-1(18),4,15(19),16-tetraen-14-one

The title compound is prepared similarly to example 9, applying in step g) the conditions described for example 13 followed by purification by preparative thin layer chromatography (DCM/MeOH/NH₃=90/9/1).

¹H-NMR (400 MHz, d6-DMSO): 8.27 (d, 1H), 7.58 (s, 1H), 7.15 (d, 1H), 7.05 (d, 1H), 6.91 (s, 1H), 6.80 (s, 1H), 6.71 (s, 1H), 5.93-5.84 (m, 1H), 5.76-5.72 (m, 1H), 5.59-5.52 (m, 1H), 4.30 (s, 2H), 4.15-3.77 (m, 4H), 3.61-3.46 (m, 4H), 3.38-3.33 (m, 2H), 3.30 (s, 3H), 2.74-2.64 (m, 2H), 1.65-1.11 (m, 7H), 1.28 (s, 9H), 1.02-0.95 (m, 2H), 0.87 (d, 3H).

### Building Block A1

### 3-(Allyl-benzyloxycarbonyl-amino)-5-methoxymethyl-benzoic acid

### a) 3-Hydroxymethyl-5-nitro-benzoic acid methyl ester

Monomethyl-5-nitroisophthalate (22.5 g, 100 mmol, 1 eq) and triethylamine (16.7 ml, 120 mmol, 1.2 eq) are dissolved in THF (200 ml) and stirred at 0° C. Isopropylchloroformate in toluene (140 ml, 1 N in toluene, 140 mmol, 1.4 eq) is added within 30 min. After stirring for 90 min at 0° C., the reaction mixture is poured on ice and 50 ml of 0.1 M aqueous HCl, and then diluted with TBME. The organic layer is separated, dried with sodium sulfate, filtered and concentrated. The crude product is dissolved in 300 ml of THF and stirred at room temperature. Sodium borohydride (12.5 g, 330 mmol, 3.3 eq) is dissolved in 100 ml of ice water and added within 15 min. The reaction is stirred for 1 hour at room temperature, then the mixture is diluted with TBME and water. The organic layer is washed with brine, dried with sodium sulfate, filtered and concentrated to give the product.

1H-NMR (400 MHz, CDCl3): 8.80 (s, 1H), 8.48 (s, 1H), 8.39 (s, 1H), 4.93 (s, 2H), 4.01 (s, 3H).

### b) 3-Methoxymethyl-5-nitro-benzoic acid methyl ester

3-Hydroxymethyl-5-nitro-benzoic acid methyl ester (8.0 g, 37.9 mmol, 1 eq) was dissolved in 80 ml of DMF. Sodium hydride (2.15 g, 49.3 mmol, 1.3 eq) was added at 0° C. The suspension was stirred for 30 min at room temperature, then methyliodide (4.57 ml, 49.3 mmol, 1.3 eq) was added. The reaction was stirred for 3 hours at room temperature and was then quenched by the addition of 1 M HCl and TBME. The organic layer was dried with sodium sulfate, filtered and concentrated. The residue was purified by column chromatography using EtOAc/hexane in a ratio of 1 to 3 to give the product.

1H-NMR (400 MHz, CDCl3): 8.80 (s, 1H), 8.43 (s, 1H), 8.38 (s, 1H), 4.61 (s, 2H), 4.00 (s, 3H), 3.52 (s, 3H).

### c) 3-Benzyloxycarbonylamino-5-methoxymethyl-benzoic acid methyl ester

3-Methoxymethyl-5-nitro-benzoic acid methyl ester (3.80 g, 16.9 mmol, 1 eq) is dissolved in EtOH (80 ml). Tin(II)chloride dihydrate (1.58 g, 7 mmol, 7 eq) is added and the reaction is heated to 75° C. for 90 min. The reaction mixture is diluted with EtOAc and aqueous sodium bicarbonate, the organic layer is separated, dried with sodium sulfate, filtered and concentrated to give a residue. The crude product is dissolved in THF, and CbzCl (0.4 ml, 1.30 mmol, 1.2 eq) is added to the reaction mixture, followed by aqueous sodium bicarbonate. The reaction mixture is stirred for 1 hour at room temperature. The organic layer is diluted with EtOAc, separated, dried with sodium sulfate, filtered and concentrated. The residue is purified by column chromatography using EtOAc/hexane in a ratio of 1 to 4 to give the product.

1H-NMR (400 MHz, CDCl3): 7.94 (s, 1H), 7.84-7.70 (m, 2H), 7.46-7.38 (m, 5H), 6.82 (s, 1H), 5.25 (s, 2H), 4.52 (s, 2H), 3.93 (s, 3H), 3.42 (s, 3H).

### d) 3-(Allyl-benzyloxycarbonyl-amino)-5-methoxymethyl-benzoic acid methyl ester

3-Benzyloxycarbonylamino-5-methoxymethyl-benzoic acid methyl ester (1.98 g, 6 mmol, 1 eq) is dissolved in 25 ml of DMF. Sodium hydride (327 mg, 55%, 7.5 mmol, 1.25 eq) is added to the reaction mixture, and the mixture is stirred for 40 min at 0° C. Allyl bromide (653 μl, 7.5 mmol, 1.25 eq) is added, and the reaction mixture is stirred for 30 min at room temperature. The mixture is then poured on ice water and extracted with EtOAc. The organic layer is separated, dried with sodium sulfate, filtered and concentrated. The residue is purified by column chromatography using EtOAc/hexane in a ratio of 1 to 4 to give the product.

1H-NMR (400 MHz, CDCl3): 7.92-7.88 (m, 2H), 7.48 (s, 1H), 7.40-7.30 (m, 5H), 6.00-5.87 (m, 1H), 5.20-5.17 (m, 4H), 4.50 (s, 2H), 4.34 (d, 2H), 3.94 (s, 3H), 3.40 (s, 3H).

### e) 3-(Allyl-benzyloxycarbonyl-amino)-5-methoxymethyl-benzoic acid

3-(Allyl-benzyloxycarbonyl-amino)-5-methoxymethyl-benzoic acid methyl ester (1.10 g, 2.68 mmol, 1 eq) is dissolved in methanol (40 ml) and 1 N aqueous lithium hydroxide (6 ml). The reaction is stirred for 1 hour at room temperature. The reaction mixture is then diluted with 1 M aq. HCl and DCM, the combined organic solvents are separated and washed with brine, dried with magnesium sulfate, filtered and concentrated to give the product.

1H-NMR (400 MHz, CDCl3): 7.94 (s, 2H), 7.55 (s, 1H), 7.40-7.20 (m, 5H), 6.00-5.88 (m, 1H), 5.22-5.18 (m, 4H), 4.53 (s, 2H), 4.37 (d, 2H), 3.40 (s, 3H).

### Building Block A2

### 3-(Benzyloxycarbonyl-pent-4-enyl-amino)-5-methoxymethyl-benzoic acid

The title compound is prepared similarly to building block A1, using 5-bromo-pent-1-ene instead of allyl bromide in step d).

¹H-NMR (400 MHz, d6-DMSO): 7.70 (br s, 1H), 7.64 (br s, 1H), 7.33-7.22 (m, 6H), 5.77-5.66 (m, 1H), 5.07 (s, 2H), 4.95-4.87 (m, 2H), 4.42 (s, 2H), 3.64 (t, 2H), 1.97 (q, 2H), 1.57-1.50 (m, 2H).

### Building Block A3

### 3-(Benzyloxycarbonyl-pent-4-enyl-amino)-5-oxazol-2-yl-benzoic acid

### a) 3-Nitro-5-oxazol-2-yl-benzoic acid methyl ester

To a suspension of 20 g (87.9 mmol mono-methyl-5-nitroisophthalate in 300 ml toluene are added 300 μl DMF and 12.93 ml (175.9 mmol) thionylchloride and the reaction mixture is stirred at 80° C. for 7 hours. The reaction mixture is concentrated to give white crystals. The crystals are dissolved in 200 ml sulfolan, then 13.4 g (194 mmol) triazole is added, followed by 12.3 g (88.0 mmol) potassium carbonate. The reaction mixture is stirred at 90° C. for 16 hours. The reaction mixture is then filtered and diluted with diethyl ether and 0.1 N aq. HCl solution. The organic layer is washed with water, dried with sodium sulfate, filtered and concentrated. The residue is purified by column chromatography using acetone and hexane in a ratio 1/6 to give the product.

¹H-NMR (400 MHz, CDCl₃): 9.10 (s, 1H), 9.04 (s, 1H), 8.93 (s, 1H), 7.83 (s, 1H), 7.39 (s, 1H), 4.03 (s, 3H).

### b) 3-Nitro-5-oxazol-2-yl-benzoic acid

3-Nitro-5-oxazol-2-yl-benzoic acid methyl ester (2.50 g, 10.0 mmol, 1 eq) is dissolved in MeOH (130 ml), THF (50 ml) and water (40 ml). Lithium hydroxide monohydrate (3.25 g, 76.7 mmol, 7.69 eq) is added and the reaction mixture is stirred at room temperature over night. The reaction mixture is diluted with EtOAc and aq. 1 N HCl solution, the organic layer is washed with brine, dried with sodium sulfate, filtered and concentrated to give the product.

¹H-NMR (400 MHz, d6-DMSO): 8.83 (s, 1H), 8.80 (s, 1H), 8.70 (s, 1H), 8.40 (s, 1H), 7.58 (s, 1H).

### c) 3-Amino-5-oxazol-2-yl-benzoic acid

3-Nitro-5-oxazol-2-yl-benzoic acid (1 g, 4.23 mmol, 1 eq) is dissolved in a mixture of MeOH (50 ml) and THF (25 ml). Pd on charcoal is added (100 mg, Engelhard 4505) and the reaction is stirred for 4 hours at room temperature at 1 bar of hydrogen. The reaction mixture is filtered and concentrated to give the product.

¹H-NMR (400 MHz, d6-DMSO): 8.20 (s, 1H), 7.70 (s, 1H), 7.41 (s, 1H), 7.39 (s, 1H), 7.30 (s, 1H), 5.70 (bs, 2H).

### d) 3-Benzyloxycarbonylamino-5-oxazol-2-yl-benzoic acid

3-Amino-5-oxazol-2-yl-benzoic acid (800 mg, 3.38 mmol, 1 eq) is suspended in THF (50 ml). Carbobenzoxychloride (1.47 ml, 50%, 4.40 mmol, 1.3 eq) in toluene is added, followed by saturated aq. sodium bicarbonate. The reaction is stirred at room temperature for 20 hours. Aqueous 2 N HCl and EtOAc are added and the layers separated. The organic layer is washed with brine, dried with sodium sulfate, filtered and concentrated. The residue is purified by column chromatography using EtOAc/hexane/AcOH in a ratio of 50/49/1 to give the product.

MS: 339 (M+H)⁺, 337 (M−H)⁺

### e) 3-Benzyloxycarbonylamino-5-oxazol-2-yl-benzoic acid methyl ester

To the solution of thionylchloride (2.11 ml, 28.7 mmol, 7 eq) in MeOH (20 ml) and THF (10 ml) is added slowly at 0° C. the solution of 3-benzyloxycarbonylamino-5-oxazol-2-yl-benzoic acid (1.4 g, 4.10 mmol, 1 eq) in MeOH (10 ml). The reaction mixture is stirred for 20 hours and then diluted with EtOAc and aq. sodium bicarbonate. The organic layer is dried with sodium sulfate, filtered and concentrated to give the product.

MS: 353 (M+H)⁺, 351 (M−H)⁺

### f) 3-(Benzyloxycarbonyl-pent-4-enyl-amino)-5-oxazol-2-yl-benzoic acid methyl ester

A mixture of 0.2 g (0.57 mmol) 3-benzyloxycarbonylamino-5-oxazol-2-yl-benzoic acid methyl ester, 0.158 mg (1.14 mmol) potassium carbonate and 0.17 ml (1.14 mmol) 5-bromo-1-pentene in 3 ml DMF are stirred for 16 h. Water is added and the mixture extracted with EtOAc. The organic phase is washed with water, dried with sodium sulfate and chromatographed on silica gel (hexanes/EtOAc 4:1)

MS (ES+): 421=[M+H]⁺

### g) 3-(Benzyloxycarbonyl-pent-4-enyl-amino)-5-oxazol-2-yl-benzoic acid

A solution of 3.3 g (7.87 mmol) 3-(Benzyloxycarbonyl-pent-4-enyl-amino)-5-oxazol-2-yl-benzoic acid methyl ester in 30 ml MeOH is treated with 15.7 ml 1 N sodium hydroxide. When the starting material has disappeared the mixture is neutralized with 1 N HCl (pH 3) and extracted with DCM. The combined organic extracts are dried with sodium sulfate and evaporated.

¹H-NMR (400 MHz, CDCl3): 8.79 (s, 1H), 8.19 (s, 1H), 8.07 (s, 1H), 7.80 (s, 1H), 7.40-7.26 (m, 5H), 5.83-5.72 (m, 1H), 5.21 (s, 2H), 5.03-4.95 (m, 2H), 3.81 (t, 2H), 2.15-2.06 (m, 2H), 1.77-1.70 (m, 2H).

### Building Block A4

### 3-Oxazol-2-yl-5-pent-4-enyloxy-benzoic acid

### a) 5-Pent-4-enyloxy-isophthalic acid dimethyl ester

To a solution of 5-hydroxy-isophthalic acid dimethyl ester in 200 ml acetone are added 17.97 g (130 mmol) potassium carbonate and 12.51 ml (17.88 g, 120 mmol) 5-bromo-1-pentene, the mixture is heated to reflux for 16 h. Additional 6.25 (8.94 g, 60 mmol) 5-bromo-1-pentene and 9.67 g (70 mmol) potassium carbonate are added and refluxing is continued for 8 h. To the mixture are added 130 ml DCM, 130 ml 1 M HCl and the layers are separated. The aqueous phase is extracted with DCM, the combined organic layers are washed with half-saturated aqueous sodium chloride solution, dried with sodium sulfate and evaporated to yield the product as yellowish oil which is used for the next step without further purification.

¹H-NMR (400 MHz, d6-DMSO): 8.03 (s, 1H), 7.63 (s, 2H), 5.90-5.80 (m, 1H), 5.06-4.96 (m, 2H), 4.07 (t, 2H), 3.86 (s, 6H), 2.18 (q, 2H), 1.85-1.79 (m, 2H).

### b) 5-Pent-4-enyloxy-isophthalic acid monomethyl ester

To a solution of 20.6 g (74 mmol) 5-pent-4-enyloxy-isophthalic acid dimethyl ester in 243 ml THF/MeOH (1/2) are added at 0° C. 81 ml (81 mmol) aqueous 1 M sodium hydroxide the mixture is stirred at 0° C. for 2 h and at rt for 2 h. The reaction mixture is acidified to pH 3 by adding 85 ml 1 M HCl and the organic solvents are evaporated. The residual solution is extracted with TBME and DCM, the combined organic layers are dried with sodium sulfate and evaporated. The residue is purified by chromatography on silica gel (DCM/MeOH 98/2 to 80/20) and gives the product as colorless solid.

¹H-NMR (400 MHz, d6-DMSO): 8.04 (t, 1H), 7.64-7.63 (m, 1H), 7.60-7.59 (m, 1H), 5.90-5.80 (m, 1H), 5.07-4.96 (m, 2H), 4.07 (t, 2H), 3.86 (s, 3H), 2.18 (q, 2H), 1.86-1.79 (m, 2H).

### c) N-(2,2-Dimethoxy-ethyl)-5-pent-4-enyloxy-isophthalamic acid methyl ester

To a solution of 6.61 g (25 mmol) 5-pent-4-enyloxy-isophthalic acid monomethyl ester in 250 ml DCM are added 2.41 ml (3.56 g, 27.5 mmol) oxalyl chloride and 0.01 ml DMF, the mixture is stirred at rt for 4 h. A solution of 3.06 ml (2.98 g, 27.5 mmol) aminoacetaldehyde dimethyl acetal in 50 ml DCM is added at 0° C. followed by 165 ml aqueous 1 M sodium carbonate solution and stirring is continued at rt for 1 h. To the reaction mixture are added 125 ml saturated aqueous sodium chloride solution, the layers are separated, the aqueous phase extracted with DCM, the combined organic layers dried with sodium sulfate and evaporated. The residue is purified by chromatography on silica gel (DCM/MeOH 99/1 to 95/5) and gives the product as colorless oil.

¹H-NMR (400 MHz, d6-DMSO): 8.74 (t, 1H), 8.01 (t, 1H), 7.64 (dd, 1H), 7.54 (dd, 1H), 5.91-5.81 (m, 1H), 5.08-4.96 (m, 2H), 4.51 (t, 1H), 4.07 (t, 2H), 3.87 (s, 3H), 3.35 (t, 2H), 3.28 (s, 6H), 2.19 (q, 2H), 1.86-1.80 (m, 2H).

### d) N-(2-Oxo-ethyl)-5-pent-4-enyloxy-isophthalamic acid methyl ester

To a solution of 5.2 g (14.8 mmol) N-(2,2-dimethoxy-ethyl)-5-pent-4-enyloxy-isophthalamic acid methyl ester in 29.6 ml THF are added 14.8 ml 2 M HCl and the mixture is stirred at rt for 7 h, followed by 30 min at 50° C. At rt 150 ml DCM are added, the layers separated, the aqueous phase extracted with DCM and the combined organic layers dried with sodium sulfate and evaporated. This yields the product as thick oil which is used for the next step without further purification.

¹H-NMR (400 MHz, d6-DMSO): 9.51 (s, 1H), 9.10 (t, 1H), 8.05 (t, 1H), 7.68 (dd, 1H), 7.57 (dd, 1H), 5.91-5.81 (m, 1H), 5.07-4.97 (m, 2H), 4.12-4.04 (m, 1H), 3.87 (s, 3H), 3.84 (t, 2H), 2.19 (q, 2H), 1.87-1.78 (m, 2H).

### e) 3-Oxazol-2-yl-5-pent-4-enyloxy-benzoic acid methyl ester

To a solution of 4.71 g (14.8 mmol) N-(2-oxo-ethyl)-5-pent-4-enyloxy-isophthalamic acid methyl ester in 220 ml AcCN are added 7.36 g (29.5 mmol) hexachloroethane, 7.86 g (29.5 mmol) triphenylphosphine, 4.23 ml (4.15 g, 59.1 mmol) pyridine and the mixture is stirred at rt for 16 h. After adding 450 ml DCM and 300 ml saturated aqueous sodium chloride solution the layers are separated, the aqueous layer extracted with DCM, the combined organic layers dried with sodium sulfate and evaporated. The residue is purified by chromatography on silica gel (cyclohexane/EtOAc 90/10) and gives the product as colorless oil.

¹H-NMR (400 MHz, d6-DMSO): 8.25 (s, 1H), 8.09-8.07 (m, 1H), 7.69-7.68 (m, 1H), 7.54-7.52 (m, 1H), 7.41 (s, 1H), 5.91-5.81 (m, 1H), 5.07-4.97 (m, 2H), 4.11 (t, 2H), 3.88 (s, 3H), 2.20 (q, 2H), 1.88-1.81 (m, 2H).

### f) 3-Oxazol-2-yl-5-pent-4-enyloxy-benzoic acid

To a solution of 1.37 g (4.77 mmol) 3-oxazol-2-yl-5-pent-4-enyloxy-benzoic acid methyl ester in 20.8 ml THF/MeOH (1/1) are added at 0° C. 5.2 ml aqueous 1 M sodium hydroxide and the mixture is stirred for 72 h and allowed to warm to rt. The organic solvents are evaporated, the residual aqueous solution is washed with TBME, acidified to pH 2 by adding 1 M HCl and extracted with DCM/EtOH (80/20). The combined organic layers are dried with sodium sulfate and evaporated to yield the product as colorless solid.

¹H-NMR (400 MHz, d6-DMSO): 13.37 (br, 1H), 8.23 (d, 1H), 8.07 (t, 1H), 7.63 (dd, 1H), 7.52 (dd, 1H), 7.40 (d, 1H), 5.91-5.81 (m, 1H), 5.08-4.97 (m, 2H), 4.10 (t, 2H), 2.19 (q, 2H), 1.88-1.81 (m, 2H).

### Building Block A5

### 5-(Benzyloxycarbonyl-pent-4-enyl-amino)-N,N-dimethyl-isophthalmic acid

### a) 5-Benzyloxycarbonylamino-isophthalic acid monomethylester

Monomethyl-5-nitroisophthalate (50 g, 220 mmol, 1 eq) is dissolved in a mixture of 650 ml of MeOH and 350 ml of THF. 3 g of Pd/C are added, and the reaction is hydrogenated over night under 1 bar of hydrogen. The reaction mixture is then filtered and concentrated to give the amine as a crude product, which is then dissolved in a mixture of THF (200 ml) and aqueous sodium bicarbonate (400 ml). CbzCl (62 ml, 50% in toluene, 184 mmol, 0.9 eq) are added to the reaction mixture, and the reaction is stirred for 1 hour. CbzCl (31 ml, 50% in toluene, 92 mmol, 0.45 eq) are added, and the reaction is stirred over night. The white solid which crashes out, is washed with water and diethyl ether to give the product.

1H-NMR (400 MHz, dmso-d6): 8.40 (s, 1H), 8.38 (s, 1H), 8.17 (s, 1H), 7.50-7.37 (m, 5H), 5.21 (s, 2H), 3.92 (s, 3H).

### b) 5-Benzyloxycarbonylamino-N,N-dimethyl-isophthalamic acid methyl ester

To 10 ml thionylchloride are added 3.29 g (9.99 mmol) 5-benzyloxycarbonylamino-isophthalic acid monomethylester and the mixture is heated to reflux for 1 h, excess thionylchloride is evaporated and the residue is dissolved in 20 ml DCM. At 0° C. a solution of 1.36 g (30 mmol) dimethylamine in 30 ml THF is added dropwise then the mixture is stirred at rt for 1 h. To the reaction mixture are added 80 ml DCM and 100 ml half-saturated aqueous ammonium chloride solution. The layers are separated, the aqueous layer is extracted with DCM, the combined organic layers are washed with water, dried with sodium sulfate and evaporated. The residue is purified twice by chromatography on silica gel (cyclohexane/EtOAc 80/20 to EtOAc) and gives the product as colorless oil.

¹H-NMR (400 MHz, d6-DMSO): 10.13 (s, 1H), 8.16 (t, 1H), 7.69 (t, 1H), 7.52 (t, 1H), 7.42-7.30 (m, 5H), 5.16 (s, 2H), 3.84 (s, 3H), 2.97 (br s, 3H), 2.86 (br s, 3H).

### c) 5-(Benzyloxycarbonyl-pent-4-enyl-amino)-N,N-dimethyl-isophthalamic acid methyl ester

To a solution of 803 mg (2.25 mmol) 5-benzyloxycarbonylamino-N,N-dimethyl-isophthalamic acid methyl ester in 4.5 ml DMF are added at 0° C. 177 mg (4.06 mmol) sodium hydride (60% in oil) and 0.412 ml (519 mg, 3.38 mmol) 5-bromo-1-pentene, the mixture is allowed to warm to rt and stirred at rt for 2 h. To the reaction mixture are added 45 ml toluene and 45 ml saturated aqueous ammonium chloride solution, the layers are separated and the aqueous layer is extracted with toluene. The combined organic layers are washed with water, dried with sodium sulfate and evaporated. The residue is purified twice by chromatography on silica gel (cyclohexane/EtOAc 90/10 to 50/50) and gives the product as colorless resin.

¹H-NMR (400 MHz, d6-DMSO): 7.86 (t, 1H), 7.76 (t, 1H), 7.61 (t, 1H), 7.33-7.23 (m, 5H), 5.76-5.66 (m, 1H), 5.09 (s, 2H), 4.95-4.87 (m, 2H), 3.86 (s, 3H), 3.71 (t, 2H), 2.97 (br s, 3H), 2.81 (br s, 3H), 2.01-1.95 (m, 2H), 1.58-1.51 (m, 2H).

### d) 5-(Benzyloxycarbonyl-pent-4-enyl-amino)-N,N-dimethyl-isophthalmic acid

To a solution of 509 mg (1.20 mmol) 5-(benzyloxycarbonyl-pent-4-enyl-amino)-N,N-dimethyl-isophthalamic acid methyl ester in 7.2 ml THF/MeOH (1/1) are added at 0° C. 1.8 ml aqueous 1 M sodium hydroxide and the mixture is stirred at rt for 3 h. The mixture is acidified to pH 3 by adding 1 M HCl and the organic solvents are evaporated. The residual aqueous solution is extracted with DCM/EtOH (80/20), the combined organic layers are washed with water, dried with sodium sulfate and evaporated to give the product as colorless solid.

¹H-NMR (400 MHz, d6-DMSO): 13.32 (br, 1H), 7.83 (t, 1H), 7.74 (t, 1H), 7.53 (br s, 1H), 7.33-7.23 (m, 5H), 5.77-5.67 (m, 1H), 5.09 (s, 2H), 4.95-4.87 (m, 2H), 3.69 (t, 2H), 2.96 (br s, 3H), 2.81 (br s, 3H), 1.98 (q, 2H), 1.58-1.51 (m, 2H).

### Building Block A6

### N,N-Dimethyl-5-pent-4-enyloxy-isophthalmic acid

### a) N,N-Dimethyl-5-pent-4-enyloxy-isophthalamic acid methyl ester

To 12.6 ml thionylchloride are added 3.33 g (12.5 mmol) 5-pent-4-enyloxy-isophthalic acid monomethyl ester (see building block A4) and the mixture is heated to reflux for 1 h, excess thionylchloride is evaporated and the residue is dissolved in 26 ml DCM. At 0° C. a solution of 1.72 g (37.8 mmol) dimethylamine in 38 ml THF is added dropwise then the mixture is stirred at rt for 1 h. To the reaction mixture are added 80 ml DCM and 100 ml half-saturated aqueous ammonium chloride solution. The layers are separated, the aqueous layer is extracted with DCM, the combined organic layers are washed with water, dried with sodium sulfate and evaporated. The residue is purified by chromatography on silica gel (DCM/MeOH 99.5/0.5 to 95/5) and gives the product as colorless oil.

¹H-NMR (400 MHz, d6-DMSO): 7.47-7.45 (m, 2H), 7.20 (dd, 1H), 5.91-5.81 (m, 1H), 5.08-4.97 (m, 2H), 4.06 (t, 2H), 3.86 (s, 3H), 2.98 (br s, 3H), 2.88 (br s, 3H), 2.18 (q, 2H), 1.86-1.79 (m, 2H).

### b) N,N-Dimethyl-5-pent-4-enyloxy-isophthalmic acid

To a solution of 2.2 g (7.57 mmol) N,N-dimethyl-5-pent-4-enyloxy-isophthalamic acid methyl ester 5 in 16.6 ml THF/MeOH (1/1) are added at 0° C. 8.3 ml aqueous 1 M sodium hydroxide and the mixture is stirred at rt for 3 h. The mixture is acidified to pH 3 by adding 1 M HCl and the organic solvents are evaporated. The residual aqueous solution is extracted with DCM, the combined organic layers are washed with half-saturated aqueous sodium chloride solution, dried with sodium sulfate and evaporated to give the product as colorless solid.

¹H-NMR (400 MHz, d6-DMSO): 13.18 (br, 1H), 7.44 (s, 1H), 7.43 (s, 1H), 7.14 (t, 1H), 5.90-5.80 (m, 1H), 5.07-4.95 (m, 2H), 4.04 (t, 1H), 2.97 (br s, 3H), 2.88 (br s, 3H), 2.17 (q, 2H), 1.85-1.78 (m, 2H).

### Building Block A7

### 2-Chloro-6-pent-4-enyloxy-isonicotinic acid

To a solution of 2.35 g (12.0 mmol) 2,6-dichloroisonicotinic acid in 25 ml 4-penten-1-ol are added in portions 1.1 g (25.2 mmol) sodium hydride (55%) and the mixture is heated to 120° C. for 17 h. Additional 314 mg (7.2 mmol) sodium hydride (55%) are added and after 7 h at 120° C., 157 mg (3.6 mmol) of sodium hydride (55%) are added and stirring is continued at 120° C. for 16 h. After cooling the reaction mixture to rt 192 ml water are added slowly and the mixture is extracted with TBME. The aqueous phase is acidified with 15.6 ml 4 M HCl to pH 1 and extracted with EtOAc. The combined organic layers are dried with sodium sulfate and evaporated. The residue is purified by chromatography on silica gel (DCM/MeOH/NH3 85/13.5/1.5) and yields the product as brownish foam.

¹H-NMR (400 MHz, d6-DMSO): 7.22 (s, 1H), 7.18 (br, 1H), 6.97 (s, 1H), 5.90-5.76 (m, 1H), 5.07-4.93 (m, 2H), 4.18 (t, 2H), 2.15 (q, 2H), 1.82-1.75 (m, 2H).

### Building Block A8

### 3-Methoxy-5-pent-4-enylamino-benzoic acid

### a) 3-Methoxy-5-nitro-benzoic acid methyl ester

To a solution of 12.82 g (68.6 mmol) 3-hydroxy-5-nitrobenzoic acid in 70 ml DMF are added 28.7 g (206 mmol) powdered potassium carbonate, the mixture is cooled to 0° C. and 9.46 ml (151 mmol) methyl iodide are added. The reaction mixture is allowed to warm to rt and stirring is continued for 16 h. 350 ml water are added and the mixture is extracted with toluene. The combined organic layers are washed with water, dried with sodium sulfate and evaporated to yield the product as yellow solid.

¹H-NMR (400 MHz, d6-DMSO): 8.19 (dd, 1H), 7.95 (t, 1H), 7.81 (q, 1H), 3.94 (s, 3H), 3.91 (s, 3H).

### b) 3-Amino-5-methoxy-benzoic acid methyl ester

A solution of 13.2 g (61.0 mmol) 3-methoxy-5-nitro-benzoic acid methyl ester in 915 ml MeOH is stirred at rt in the presence of 2.64 g 10% Pd/C under a hydrogen atmosphere for 3 h. The catalyst is filtered off and the filtrate evaporated to give the product as colorless solid.

¹H-NMR (400 MHz, d6-DMSO): 6.81 (t, 1H), 6.61 (dd, 1H), 6.35 (t, 1H), 5.38 (s, 1H), 3.78 (s, 3H), 3.69 (s, 3H).

### c) 3-Methoxy-5-pent-4-enylamino-benzoic acid methyl ester

To a solution of 544 mg (3.0 mmol) 3-amino-5-methoxy-benzoic acid methyl ester in 30 ml MeOH are added 0.035 ml (0.6 mmol) glacial acetic acid and 0.367 ml (3.6 mmol) 4-pentenal. After stirring for 15 min at rt the mixture is cooled to 0° C. and 273 mg (3.9 mmol) sodium cyanoborohydride are added, stirring is continued for 16 h allowing the reaction mixture to warm to rt. By adding 1 M HCl the pH is adjusted to 7, the organic solvent is evaporated and the mixture is extracted with DCM. The combined organic layers are washed with half-saturated sodium chloride solution, dried with sodium sulfate and evaporated. The residue is purified by chromatography on silica gel (DCM) and gives the product as colorless solid.

¹H-NMR (400 MHz, d6-DMSO): 6.79 (t, 1H), 6.63 (dd, 1H), 6.31 (t, 1H), 5.94 (t, 1H), 5.89-5.79 (m, 1H), 5.06-4.95 (m, 2H), 3.79 (s, 3H), 3.72 (s, 3H), 3.00 (q, 2H), 2.12 (q, 2H), 1.66-1.58 (m, 2H).

### d) 3-Methoxy-5-pent-4-enylamino-benzoic acid

To a solution of 420 mg (1.68 mmol) 3-methoxy-5-pent-4-enylamino-benzoic acid methyl ester in 11 ml THF/MeOH (1/1) are added at 0° C. 3.7 ml (3.7 mmol) aqueous 1 M sodium hydroxide, while stirring for 16 h the mixture is allowed to warm to rt. By adding 1 M HCl the pH is adjusted to 3, the organic solvents are evaporated and the mixture is extracted with DCM. The combined organic layers are washed with half-saturated sodium chloride solution, dried with sodium sulfate and evaporated to give the product as yellowish solid.

¹H-NMR (400 MHz, d6-DMSO): 12.64 (br s, 1H), 6.76 (t, 1H), 6.62 (dd, 1H), 6.26 (t, 1H), 5.88-5.77 (m, 2H), 5.05-4.94 (m, 2H), 3.69 (s, 3H), 2.99 (q, 2H), 2.10 (q, 2H), 1.65-1.57 (m, 2H).

### Building Block A9

### 2-(Acetyl-allyl-amino)-6-methyl-isonicotinic acid

### a) 2-(N′-Isopropylidene-hydrazino)-6-methyl-isonicotinic acid ethyl ester

A mixture of 7.35 g (42.86 mmol) 2-chloro-6-methyl-isonicotinic acid, 10.75 g (250 mmol) hydrazine hydrate and 10.7 ml aqueous 4 N sodium hydroxide is stirred at 125° C. for 24 h. The mixture is evaporated to dryness, taken up in 35 ml water, 35 ml EtOH and 50 ml acetone and stirred for 1 h. The mixture is concentrated once more and refluxed in a solution of 20 ml thionylchloride in 200 ml EtOH. After 1.5 h the mixture is cooled down and filtered. The filtrate is diluted with ethyl acetate and washed with 10% aq. sodium bicarbonate solution. The aqueous phase is extracted with EtOAc/acetone (4:1) three times. The combined organic layers are dried with sodium sulfate and chromatographed on silica gel (EtOAc/hexanes=1:2) to give a brownish oil, which crystallizes from EtOH/water.

¹H-NMR (400 MHz, CDCl₃): 8.05 (br, 1H), 7.59 (s, 1H), 7.14 (s, 1H), 4.39 (q, 2H), 2.46 (s, 3H), 2.07 (s, 3H), 1.93 (s, 3H), 1.41 (t, 3H).

### b) 2-Amino-6-methyl-isonicotinic acid ethyl ester

A solution of 8.37 g (35.6 mmol) 2-(N′-isopropylidene-hydrazino)-6-methyl-isonicotinic acid ethyl ester in 150 ml EtOH is hydrogenated for 11 h at 80° C. and 6 bar hydrogen in the presence of 25 g Raney-Ni. After cooling down the mixture is filtered over celite and evaporated. The product is crystallized from EtOH/water to give white crystals.

¹H-NMR (400 MHz, CDCl₃): 7.08 (s, 1H), 6.93 (s, 1H), 4.61 (br, 2H), 4.19 (q, 2H), 2.46 (s, 3H), 1.41 (t, 3H).

### c) 2-Acetylamino-6-methyl-isonicotinic acid ethyl ester

A mixture of 4.50 g (25 mmol) 2-amino-6-methyl-isonicotinic acid ethyl ester, 30 ml acetic anhydride and 40 ml pyridine is stirred for 60 h. The mixture is evaporated and the title compound is isolated as a white solid and used without further purification.

¹H-NMR (400 MHz, CDCl₃): 8.54 (s, 1H), 8.2 (br, 1H), 7.50 (s, 1H), 4.42 (q, 2H), 2.53 (s, 3H), 2.23 (s, 3H), 1.42 (t, 3H).

### d) 2-(Acetyl-allyl-amino)-6-methyl-isonicotinic acid

A mixture of 5.0 g (22.5 mmol) 2-acetylamino-6-methyl-isonicotinic acid ethyl ester, 4.7 g (33.7 mmol) potassium carbonate and 3.8 ml (45 mmol) allyl bromide are stirred in 20 ml DMF. After 15 h the reaction is not complete according TLC analysis. Allyl bromide (1.9 ml, 22.5 mmol), ceasium carbonate (7.3 g, 22.5 mmol) and tetrabutyl ammonium iodide (8.3 g 22.5 mmol) are added and the mixture is stirred for 2 days. The mixture is diluted with water and extracted with ethyl acetate. The organic layer is washed with water, dried with sodium sulfate and chromatographed on silica gel (gradient toluene/TBME 8 to 2:1). Yield 5.79 g of ethyl ester contaminated with 10% allyl ester that could not be separated. The product is dissolved in 50 ml MeOH and treated with 26.5 ml aqueous 1 N sodium hydroxide. When the starting material has disappeared the mixture is neutralized with 1 N HCl (pH 3) and extracted with ethyl acetate. The product is evaporated and crystallized from aqueous MeOH to give the title compound as white crystals.

¹H-NMR (400 MHz, d6-DMSO): 7.73 (s, 1H), 7.59 (s, 1H), 5.93-5.82 (m, 1H), 5.16-5.07 (m, 2H), 4.52-4.46 (m, 2H), 2.53 (s, 3H), 2.07 (s, 3H).

### Building Block A10

### 2-Allylamino-6-methoxymethyl-isonicotinic acid

### a) 2-Chloro-6-methyl-1-oxy-isonicotinic acid

2-Chloro-6-methyl-isonicotinic acid (6.86 g, 40 mmol, 1 eq) is dissolved in AcOH (40 ml). 2 ml of hydrogen peroxide (35% in water) is added to the reaction mixture, and the reaction is stirred for 76 hours at 95° C. During the reaction time, 2 ml of hydrogen peroxide (35% in water) are added five times in regular intervals. The reaction mixture is concentrated and co-evaporated with toluene to give the product.

1H-NMR (400 MHz, dmso-d6): 8.05 (d, 1H), 7.96 (d, 1H), 2.46 (s, 3H).

### b) 2-Chloro-6-hydroxymethyl-isonicotinic acid

2-Chloro-6-methyl-1-oxy-isonicotinic acid (7.3 g, 39 mmol, 1 eq) is dissolved in acetic acid anhydride, and the reaction mixture is stirred at 100° C. for 2 hours. The reaction mixture is cooled then to 40° C., and water (40 ml) is added over 2 hours. The mixture is concentrated and purified by column chromatography using DCM/MeOH/AcOH in a ratio of 360 to 39 to 1 to give the acetylated product. The acetylated product was dissolved in MeOH (50 ml), and aqueous 2 N sodium hydroxide (25 ml) was added. The reaction was stirred for 4 hours and then diluted with 2 N HCl. The mixture was concentrated and then diluted with DCM. The organic layer was separated, dried with sodium sulfate, filtered and concentrated to give the product.

MS (ES−): 186=[M−H]⁻

### c) 2-Chloro-6-methoxymethyl-isonicotinic acid

2-Chloro-6-hydroxymethyl-isonicotinic acid (4.6 g, 24.5 mmol, 1 eq) is dissolved in 100 ml of DMF. Sodium hydride (3.53 g, 73.5 mmol, 3 eq) is added at 0° C. The reaction mixture is stirred for 1 hour at 10° C., then methyliodide (7.63 ml, 123 mmol, 5 eq) is added within 15 min. The reaction is stirred at room temperature for 4 hours, and then it is quenched with 10 ml of aqueous 4 N sodium hydroxide. The reaction mixture is then diluted with 4 N HCl and concentrated. The residue is diluted with DCM/MeOH 9 to 1, and the organic layer is concentrated. The residue is purified by column chromatography using DCM/EtOH/AcOH in a ratio of 180 to 19 to 1 to give the product.

MS (ES+): 202=[M+H]⁺

### d) 2-Chloro-6-methoxymethyl-isonicotinic acid tert-butyl ester

2-Chloro-6-methoxymethyl-isonicotinic acid (3.48 g, 15.5 mmol, 1 eq) is dissolved in toluene (60 ml) and heated to 80° C. N,N-dimethylformamid-di-tertbutylacetal (7.53 ml, 31 mmol, 2 eq) is added in portions over 8 hours. The reaction mixture is then diluted with TBME and washed with aqueous sodium bicarbonate. The organic layer is dried with sodium sulfate, filtered and concentrated to give the product.

MS (ES+): 258=[M+H]⁺

### e) 2-Allylamino-6-methoxymethyl-isonicotinic acid tert-butyl ester

Pd(OAc)₂ (97 mg, 0.42 mmol, 0.05 eq), (+/−)-BINAP (269 mg, 0.42 mmol, 0.05 eq), sodium tertbutanolate (1.66 g, 17 mmol, 2 eq), and allylamine (784 mg, 12.7 mmol, 1.5 eq) are dissolved in toluene (80 ml) and stirred at 50° C. for 20 min. 2-Chloro-6-methoxymethyl-isonicotinic acid tert-butyl ester (1.38 g, 5.4 mmol, 1 eq) is dissolved in toluene (20 ml) and added to the reaction mixture at 50° C. within 20 min. The reaction is stirred at 50° C. for 1 h. The reaction mixture is cooled to room temperature and poured on ice and TBME (200 ml). 4 g of ammonium chloride is added, and the mixture is stirred for 20 min. The organic layer is separated, dried with sodium sulfate, filtered and concentrated to give the product.

1H-NMR (400 MHz, CDCl3): 7.18 (s, 1H), 6.87 (s, 1H), 6.02-5.92 (m, 1H), 5.37-5.19 (m, 2H), 4.88-4.82 (m, 1H), 4.47 (s, 2H), 4.01-3.97 (m, 2H), 3.50 (s, 3H), 1.62 (s, 9H).

### f) 2-Allylamino-6-methoxymethyl-isonicotinic acid

2-Allylamino-6-methoxymethyl-isonicotinic acid tert-butyl ester (270 mg, 0.97 mmol, 1 eq) is dissolved in 4 N HCl in dioxane (4.9 ml). The reaction is stirred for 83 h at room temperature. The reaction mixture is then concentrated and co-evaporated with toluene to give the product.

MS (ES+): 223=[M+H]⁺

### Building block B1

### [(1S,3S)-5-Allyloxy-1-((S)-2-chloro-1-hydroxy-ethyl)-3-methyl-pentyl]-carbamic acid tert-butyl ester

### a) 4-Allyloxy-butyric acid

A mixture of 13.77 g (160 mmol) γ-butyrolactone and 40 ml aqueous 4 N sodium hydroxide is refluxed for 10 minutes and evaporated. The residual white solid is dried at 80° C. under high vacuum. The product is taken up in 200 ml dry DMSO and subsequently 6.3 g (150 mmol) anhydrous lithium chloride and 12 g (150 mmol) lithium tert-butoxide are added. Under ice cooling 25.4 ml (300 mmol) allyl bromide are added at such a rate that the reaction temperature did not exceed 35° C. The mixture is stirred for three hours. Aqueous 2 N sodium hydroxide (300 ml) are added. After stirring for 1 h the mixture is washed with 100 ml TBME, acidified with 6 N HCl and ice and extracted with EtOAc. The organic phase is washed with water, dried with magnesium sulfate and evaporated. Distillation provides the product as a colorless liquid.

¹H-NMR (400 MHz, CDCl3): 6.00-5.87 (m, 1H), 5.30 (dt, 1H), 5.21 (dt, 1H), 4.00 (m, 2H), 3.54 (t, 2H), 2.53 (t, 2H), 1.96 (q, 2H).

### b) (R)-3-((R)-4-Allyloxy-2-methyl-butyryl)-4-isopropyl-5,5-diphenyl-oxazolidin-2-one

To a stirred solution of 13.78 g (95.66 mmol) 4-allyloxy-butyric acid in 400 ml THF at −30° C. is added 11.54 g (95.66 mmol) pivaloyl chloride and 34.7 ml (248.7 mmol) triethylamine. The mixture is stirred for 1.5 h at −20° C. and 26.9 g (95.66 mmol) (R)-4-isopropyl-5,5-diphenyl-oxazolidin-2-one is added followed by 4.66 g (110 mmol) lithium chloride. The mixture is stirred overnight while the temperature is allowed to rise slowly to 20° C. A 10% aqueous solution of ammonium chloride (300 ml) and 300 ml TBME are added. The organic phase is washed with 1N HCl, aqueous 1 N sodium hydroxide and brine, dried with magnesium sulfate and concentrated. The residue is taken up in TBME/hexanes and after stirring for 1 h 1.68 g of (R)-4-isopropyl-5,5-diphenyl-oxazolidin-2-one is removed by filtration. The product is obtained as a colorless oil.

¹H-NMR (400 MHz, CDCl3): 7.53-7.30 (m, 10H), 5.95-5.85 (m, 1H), 5.41 (d, 1H), 5.26 (dt, 1H), 5.18 (dt, 1H), 3.92 (d, 2H), 3.42 (t, 2H), 309-2.99 (m, 1H), 2.91-2.82 (m, 1H), 2.05-1.83 (m, 3H), 0.92 (d, 3H), 0.89 (d, 3H).

### c) (R)-3-((R)-4-Allyloxy-2-methyl-butyryl)-4-isopropyl-5,5-diphenyl-oxazolidin-2-one

To a solution of 34.2 g (84 mmol) (R)-3-((R)-4-allyloxy-2-methyl-butyryl)-4-isopropyl-5,5-diphenyl-oxazolidin-2-one in 250 ml THF at −70° C. is added 100 ml (100 mmol) of a 1 M solution of sodium hexamethyl disilazide in THF over a period of 30 minutes. The mixture is stirred for 1.5 h at −70° C. and 26.2 ml (420 mmol) iodomethane are added. Stirring is continued while the mixture slowly warms up without taking away the cooling bath. After 2 h the reaction is complete according to TLC analysis and poored onto 400 ml 10% aqueous ammonium chloride solution and 300 ml TBME. The organic phase is washed with 5% citric acid and extensively with water. After removal of all the solvents (R)-3-((R)-4-allyloxy-2-methyl-butyryl)-4-isopropyl-5,5-diphenyl-oxazolidin-2-one is obtained as a colorless oil, pure enough for further transformations.

¹H-NMR (400 MHz, CDCl3): 7.56-7.29 (m, 10H), 5.77-5.67 (m, 1H), 5.46 (d, 1H), 5.15 (dt, 1H), 5.09 (dt, 1H), 3.83-3.75 (m, 1H), 3.64-3.56 (m, 2H), 3.22-3.16 (m, 1H), 3.09-3.02 (m, 1H), 2.04-1.88 (m, 2H), 1.61-1.53 (m, 1H), 1.30 (d, 3H), 0.91 (d, 3H), 0.80 (d, 3H).

### d) (R)-4-Allyloxy-2-methyl-butyric acid methyl ester

To a solution of 36 g (85.5 mmol) (R)-3-((R)-4-allyloxy-2-methyl-butyryl)-4-isopropyl-5,5-diphenyl-oxazolidin-2-one in 180 ml THF and 450 ml MeOH at 10° C. is added 35.7 g (410 mmol) anhydrous lithium bromide. After 5 minutes the mixture becomes homogeneous and 13 g (85.5 mmol) DBU is added. After 5 h are added under cooling 180 ml 10% aqueous ammonium chloride solution and 500 ml water. The mixture is filtered and the filter cake is washed with water and TBME. 13.4 g of the chiral auxiliary are recovered. The filtrate is extracted with TBME twice and the combined organic layers are washed with 1 N HCl and brine. The product is dried with magnesium sulfate and distilled at 1 mm Hg, bp. 40-41° C. as a colorless liquid.

¹H-NMR (400 MHz, CDCl3): 5.99-5.87 (m, 1H), 5.29 (dt, 1H), 5.20 (dt, 1H), 3.98 (d, 2H), 3.70 (s, 3H), 3.51-3.45 (m, 2H), 2.71-2.62 (m, 1H), 2.08-1.98 (m, 1H), 1.76-1.67 (m, 1H), 1.21 (d, 3H).

### e) (R)-4-Allyloxy-2-methyl-butan-1-ol

A solution of 12.9 g (75 mmol) (R)-4-allyloxy-2-methyl-butyric acid methyl ester in 10 ml diethyl ether is added dropwise to a refluxing suspension of 2.85 g (75 mmol) lithium aluminium hydride in 100 ml diethyl ether. The mixture is stirred for 1 h at room temperature. The excess lithium aluminium hydride is destroyed by careful addition of 2.9 ml water, 2.9 ml aqueous 4 N sodium hydroxide and 6.5 ml water. After stirring for 1 h at room temperature the mixture is filtered and evaporated to give the title compound as a colorless liquid pure enough for further transformations.

¹H-NMR (400 MHz, CDCl3): 6.00-5.89 (m, 1H), 5.32 (dt, 1H), 5.22 (dt, 1H), 4.03 (d, 2H), 3.62-3.45 (m, 2H), 1.90-1.58 (m, 3H), 0.98 (d, 3H).

### f) 2-((S)-4-Allyloxy-2-methyl-butyl)-malonic acid diethyl ester

At +10° C. are added portionwise 21.9 g (115 mmol) tosyl chloride to a solution of 15.2 g (105 mmol) (R)-4-allyloxy-2-methyl-butan-1-ol in 150 ml dry pyridine. The mixture is stirred at room temperature overnight. The excess TsCl is destroyed by addition of 0.5 ml water and stirring for 1 h. The mixture is diluted with EtOAc, washed with 5% aqueous citric acid till all the pyridine is removed according to TLC analysis. Subsequently is washed with water (4×) and evaporated to give 28.35 g of the crude tosylate as a slightly colored oil. This product is taken up in 10 ml THF and added to a stirred solution of sodium diethyl malonate, prepared from 21.6 ml (142 mmol) diethyl malonate and 5.68 g (142 mmol, 60% in mineral oil) sodium hydride in 100 ml THF. To the homogeneous solution are added 1 g (2.7 mmol) tetrabutyl ammonium iodide and 35 ml DMF. The mixture is heated at 75° C. overnight. During the reaction sodium tosyl sulfonate precipitates. After cooling down the mixture is diluted with 5% ammonium chloride and extracted with EtOAc. The organic phase is washed with water, dried with magnesium sulfate and evaporated. The excess diethyl malonate is removed by distillation under high vacuum and the residue is purified by chromatography on silica gel (EtOAc/hexanes=1:20; 1:8 and 1:3) and gives the title compound as a colorless oil.

¹H-NMR (400 MHz, CDCl3): 6.00-5.89 (m, 1H), 5.29 (dt, 1H), 5.20 (dt, 1H), 4.22 (q, 4H), 3.99 (d, 2H), 3.48 (q, 2H), 2.05-1.98 (m, 1H), 1.78-1.60 (m, 2H), 1.53-1.47 (m, 1H), 1.30 (t, 6H) 0.97 (d, 3H).

### g (S)-2-Acetylamino-6-allyloxy-4-methyl-hexanoic acid ethyl ester

To a solution of 2.01 g (87.4 mmol) sodium metal in 75 ml EtOH is added 25 g (87.4 mmol) 2-((S)-4-allyloxy-2-methyl-butyl)-malonic acid diethyl ester. The mixture is cooled to −20° C. and 12.2 ml isoamyl nitrite (87.4 mmol) is added. The mixture is stirred at −10° C. till the starting material had disappeared. Water is added and the mixture is acidified with 2 N HCl to pH 5 and extracted with EtOAc. The organic phase is dried with sodium sulfate and evaporated to yield 18.2 g crude (S)-6-allyloxy-2-[(Z)-hydroxyimino]-4-methyl-hexanoic acid ethyl ester. The intermediate oxime is treated with 20 g (306 mmol) Zn powder in 250 ml AcOH. The reaction is exothermic and the temperature rises to 45° C. The mixture is stirred at room temperature overnight, filtered over celite, evaporated and treated immediately with 23 g acidic anhydride and 31 ml triethylamine. After 2 h the mixture is diluted with 200 ml EtOH/water and stirred for 1 h. The mixture is extracted with EtOAc and the organic phase is washed with 10% aqueous sodium carbonate, 5% aqueous citric acid and brine. The title compound is obtained as a 1:1 mixture of diastereomers after chromatography on silica gel (EtOAc/hexanes 1:2; 1:1).

MS (ES+): 272=[M+H]⁺

### h) (2S,4S)-2-acetylamino-6-allyloxy-4-methyl-hexanoic acid ethyl ester

A suspension of 15.87 g (58.48 mmole) (2S,4R/S)-2-acetylamino-6-allyloxy-4-methyl-hexanoic acid ethyl ester in 60 g phosphate buffer pH 7.5 is treated with 160 μl Alcalase Typ DX (Lot: PMNO466) under pH-stat conditions. When the conversion reached 49.1% thereaction mixture is adjusted to pH 8 and extracted with DCM. The organic phase is dried with magnesium sulfate and the solvent removed under reduced pressure to yield the undesired isomer as yellow oil.

(2R,4S)-2-Acetylamino-6-allyloxy-4-methyl-hexanoic acid ethyl ester

92.92% d.e. (HPLC Chiralpak AD-H 1192, 250×4.6 mm, 5 μl, Hexane/EtOH/MeOH 96/2/2, 1 ml/min) retention time=12.53 min (2R,4S), 17.63 min (2S,4S).

¹H-NMR (400 MHz, CDCl₃): 1.00 (d, 3H), 1.30 (t, 3H), 1.40-1.80 (m, 5H), 2.00 (s, 3H), 3.45 (m, 2H), 3.95 (d, 2H), 4.20 (q, 2H), 4.60 (q, 1H), 5.20 (dd, 2H), 5.90 (m, 2H), 6.10 (d, 1H).

The aqueous solution containing the product is used for the next step without further purification.

(2S,4S)-2-acetylamino-6-allyloxy-4-methyl-hexanoic acid ethyl ester

Rf: (AcCN/EtOH/acetic acid/H₂O=70/20/5/5): 0.67.

### i) (2S,4S)-6-Allyloxy-2-amino-4-methyl-hexanoic acid

To the aqueous phase containing (2S,4S)-2-acetylamino-6-allyloxy-4-methyl-hexanoic acid ethyl ester, is added CoCl₂ to a final concentration of 10⁻⁴ molar. After addition of 250 mg Acylase Amano (Lot: ACV12502) the mixture is stirred at room temperature until (2S,4S)-2-acetylamino-6-allyloxy-4-methyl-hexanoic acid ethyl ester disappeared completely. This solution is used for the next step without further purification.

Rf: (AcCN/EtOH/acetic acid/H₂O=70/20/5/5): 0.21.

### j) (2S,4S)-6-Allyloxy-2-tert-butoxycarbonylamino-4-methyl-hexanoic acid

To the aqueous solution of (2S,4S)-6-allyloxy-2-amino-4-methyl-hexanoic acid are added at 0° C. 100 ml THF followed by addition of 7.9 g (57.1 mmol) sodium carbonate and 9.4 g (43.7 mmol) Boc₂O. After stirring over night at rt THF is removed in vacuum and the aqueous reaction mixture is washed 3 times with DCM. The pH is adjusted to 3 and aqueous solution is extracted with DCM. The organic phase is dried with magnesium sulfate and the solvent removed under reduced pressure to give the product as a colorless oil.

¹H-NMR (400 MHz, CDCl₃): 1.00 (d, 3H), 1.45 (s, 9H), 1.50-1.80 (m, 4H), 3.50 (m, 2H), 4.00 (d, 2H), 4.30 (m, 1H), 5.00 (d, 1H), 5.25 (m, 2H), 5.90 (m, 1H)

### k) (2S,4S)-6-Allyloxy-2-tert-butoxycarbonylamino-4-methyl-hexanoic acid methyl ester

A solution of 5.3 g (17.2 mmol) (2S,4S)-6-allyloxy-2-tert-butoxycarbonylamino-4-methyl-hexanoic acid in 17.2 ml DMF is cooled to 0° C., 4.81 g (34.5 mmol) potassium carbonate (powdered) and 1.73 ml (3.94 g, 27.7 mmol) methyl iodide are added and the mixture is stirred for 2.5 days while warming to rt. After addition of 85 ml water and the mixture is extracted with toluene, the organic layers are washed with water, dried with sodium sulfate and evaporated to give the product as colorless oil, which is used for the next step without further purification.

¹H-NMR (400 MHz, d6-DMSO): 7.19 (d, 1H), 5.91-5.82 (m, 1H), 5.25-5.19 (m, 1H), 5.13-5.09 (m, 1H), 4.04-3.97 (m, 1H), 3.91-3.88 (m, 2H), 3.61 (s, 3H), 3.39 (t, 2H), 1.66-1.48 (m, 3H), 1.43-1.30 (m, 2H), 1.38 (s, 9H), 0.84 (d, 3H).

### l) [(1S,3S)-5-Allyloxy-1-(2-chloro-acetyl)-3-methyl-pentyl]-carbamic acid tert-butyl ester

A solution of 315 mg (1.00 mmol) (2S,4S)-6-allyloxy-2-tert-butoxycarbonylamino-4-methyl-hexanoic acid methyl ester in 10 ml THF is cooled at −78° C. and 0.30 ml (4.0 mmol) chloroiodo-methane are added. A 0.84 M THF solution of LDA (5.94 ml, 5.0 mmol) is added dropwise while the temperature of the reaction mixture is maintained below −73° C., and the mixture is stirred for additional 30 min. The reaction is carefully quenched with 1.1 ml (19.2 mmol) glacial acetic acid while the temperature is maintained below −65° C. After stirring for 15 min at −78° C. the mixture is allowed to warm to 0° C. and 15 ml of a half-saturated aqueous sodium chloride solution is added. The mixture is extracted with TBME, the organic layer washed with aqueous 1 M sodium bicarbonate and 1 M sodium sulfite, dried with sodium sulfate and evaporated. The product is used for the next step without further purification.

MS (LC/MS): 355.8=[M+Na]⁺

### m) [(1S,3S)-5-Allyloxy-1-((S)-2-chloro-1-hydroxy-ethyl)-3-methyl-pentyl]-carbamic acid tert-butyl ester

A solution of 77 mg (2.0 mmol) sodium borohydride in 22 ml EtOH is cooled to −78° C., a solution of crude 605 mg (1.00 mmol) [(1S,3S)-5-allyloxy-1-(2-chloro-acetyl)-3-methyl-pentyl]-carbamic acid tert-butyl ester in 6 ml EtOH is added dropwise, maintaining the internal temperature below −75° C. After stirring is continued at −78° C. for 30 min, 4.0 ml of 0.5 M HCl are added dropwise maintaining the internal temperature below −70° C. The mixture is allowed to warm to rt, the pH is adjusted to 7 and EtOH is evaporated. The residue is taken up EtOAc, washed with half-saturated aqueous sodium chloride solution, dried with sodium sulfate and evaporated. The residue is purified by chromatography on silica gel (cyclohexane/EtOAc 90/10 to 80/20) and gives the product as pale brown amorphous solid.

¹H-NMR (400 MHz, d6-DMSO): 6.56 (d, 1H), 5.90-5.80 (m, 1H), 5.24-5.18 (m, 2H), 5.12-5.08 (m, 1H), 3.90-3.86 (m, 2H), 3.56 (d, 1H), 3.47-3.40 (m, 2H), 3.37 (t, 2H), 1.61-1.42 (m, 2H), 1.40-1.28 (m, 4H), 1.36 (s, 9H), 0.81 (d, 3H).

The following compounds are obtained from the corresponding nitriles following analogously known procedures. The nitriles are commercially available or can be prepared following analogously known procedures.

### Building Block C1

### 1-(4-tert-Butyl-pyrid-2-yl)-cyclopropylamine

¹H-NMR (400 MHz, d6-DMSO): 8.26 (d, 1H), 7.77 (d, 1H), 7.08 (dd, 1H), 1.29 (s, 9H), 1.21-1.16 (m, 2H), 0.95-0.91 (m, 2H).

### Building Block C2

### 1-(4-Isopropyl-pyrid-2-yl)-cyclopropylamine

¹H-NMR (400 MHz, d6-DMSO): 8.23 (d, 1H), 7.61 (d, 1H), 6.95 (dd, 1H), 2.19-2.80 (m, 1H), 1.21 (d, 6H), 1.17 (q, 2H), 0.91 (q, 2H).

### Building Block C3

### 1-(3-tert-Butyl-phenyl)-cyclopropylamine

¹H-NMR (400 MHz, d6-DMSO): 7.40-7.37 (m, 1H), 7.28-7.26 (m, 2H), 7.16-7.12 (m, 1H), 1.35 (s, 9H), 1.10-1.06 (m, 2H), 1.02-0.98 (m, 2H).

### Building Block C4

### 1-(5-Bromo-pyrid-3-yl)-cyclopropylamine

¹H-NMR (400 MHz, d6-DMSO): 8.42 (t, 2H), 7.94 (t, 1H), 1.01 (d, 4H).

### Building Block C5

### 1-[5-(2,2-Dimethyl-propyl)-isoxazol-3-yl]-cyclopropylamine

### a) (Z)-2-Hydroxy-6,6-dimethyl-4-oxo-hept-2-enoic acid ethyl ester

To an ice-cooled solution of sodium ethanolate (128.5 g, 1.79 mol) in EtOH (2500 ml) under nitrogen atmosphere is added 4,4-dimethyl-pentan-2-one (195.0 g, 1.71 mol). Half an hour later, oxalic acid diethyl ester (231.5 g, 1.71 mol) is added. After being stirred at rt for 24 h, the reaction mixture is diluted with water, and acidified to pH 2.0 by 6N aq hydrochloric acid. The mixture is contracted to about 1 L and extracted with DCM. The combined extracts are washed with brine, dried over sodium sulfate, and concentrated in vacuo to yield the product as a brown liquid.

¹H-NMR (300 MHz, CDCl₃): 6.32 (s, 1H), 4.35 (q, 2H), 2.33 (s, 2H), 1.60 (t, 3H), 1.04 (s, 9H).

### b) 5-(2,2-Dimethyl-propyl)-isoxazole-3-carboxylic acid

To a solution of (Z)-2-hydroxy-6,6-dimethyl-4-oxo-hept-2-enoic acid ethyl ester (298.5 g, 1.39 mol) in EtOH (1600 ml) is added hydroxylamine hydrochloride (106.5 g, 1.53 mol) and the resulting solution is stirred at room temperature for 24 h. 2N aq sodium hydroxide (1740 ml, 3.48 mol) is added to the reaction and the resulting solution is stirred at rt for 2 h. The reaction mixture is acidified with 6N aq hydrochloric acid, concentrated to about 3 L, and extracted with EtOAc (2000 ml). The combined organic layers are washed with brine, dried over magnesium sulfate and concentrated. The resulting solid is washed with ether and dried to afford the product.

¹H-NMR (300 MHz, DMSO-d₆): 6.61 (s, 1H), 2.72 (s, 2H), 0.94 (s, 9H).

### c) 5-(2,2-Dimethyl-propyl)-isoxazole-3-carboxylic acid tert-butylamide

To a solution of 5-(2,2-dimethyl-propyl)-isoxazole-3-carboxylic acid (125.4 g, 0.685 mol) in THF (1500 ml) and MeCN (1500 ml) is added HOBT (101.75 g, 0.753 mol) and EDCl (144.3 g, 0.753 mol). After stirred 30 min, tert-butyl amine (86.7 ml, 0.821 mol) is added dropwise under nitrogen atmosphere and then the reaction is stirred at rt for 1.5 h. The solvents are evaporated under reduced pressure and the residue is taken into DCM (2000 ml). The mixture is washed with saturated aq sodium bicarbonate (500 ml×2), the organic layer is dried over sodium sulfate and concentrated. The residue is purified by chromatography on silica (DCM) to give the product as white solid.

MS (LC/MS): 239=[M+H]⁺

### d) 5-(2,2-Dimethyl-propyl)-isoxazole-3-carbonitrile

A mixture of 5-(2,2-dimethyl-propyl)-isoxazole-3-carboxylic acid tert-butylamide (58.0 g, 0.243 mol) and phosphorus (III) oxychloride (156 ml, 1.70 mol) is heated under nitrogen atmosphere at reflux temperature for 2 h. The reaction mixture is cooled to rt and concentrated to remove excess phosphorus (III) oxychloride. The residue is diluted with DCM (2000 ml) and washed with saturated aq sodium bicarbonate (500 ml×2). The organic layer is washed with brine, dried over sodium sulfate, and concentrated. The residue is purified by chromatography on silica (DCM/hexanes 1/1) to yield the target compound as yellow liquid.

¹H-NMR (300 MHz, CDCl₃): 6.36 (s, 1H), 2.74 (s, 2H), 1.00 (s, 9H).

### e) 1-[5-(2,2-Dimethyl-propyl)-isoxazol-3-yl]-cyclopropylamine

To a mixture of 5 g (30.4 mmol) of 5-(2,2-dimethyl-propyl)-isoxazole-3-carbonitrile and 10.1 ml (34.1 mmol) of titanium(IV) isopropoxide in 150 ml of dry diethyl ether a solution of 22 ml of ethylmagnesium bromide (3 M in diethyl ether, 66.0 mmol) is added at −70° C. The reaction mixture is allowed to reach rt within two hours, 7.6 ml (60.6 mmol) of boron trifluoride-diethyl etherate are added and stirring is continued for one hour. After addition of 90 ml of 1 M aq hydrochloric acid and 450 ml of diethyl ether two clear phases are obtained which are treated with 300 ml of 10% aq sodium hydroxide. The aqueous phase is extracted with diethyl ether, the combined organic phases are dried over sodium sulfate and evaporated to afford a dark orange oil. After filtration over a C18-bond elut column (Varian) with THF/MeCN the oil is purified by HPLC (dissolved in 6 ml of tetrahydrofuran, 25 injections, XBridge C18 column, 19×150 mm, 5 μM, gradient of 95% MeCN in water to 10% MeCN in water, containing 0.02% of ammonium hydroxide). The combined product fractions are concentrated and the product is extracted with DCM to yield the product as an orange solid.

¹H-NMR (360 MHz, CDCl₃): 5.50 (s, 1H), 2.50 (s, 2H), 1.80 (br s, 2H), 1.10-1.05 (m, 2H), 0.95-0.90 (m, 2H), 0.90 (s, 9H).

1. A compound of the formula 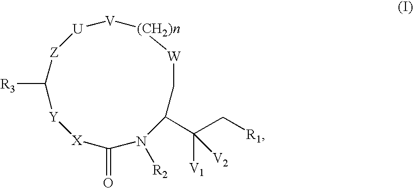
in which
R₁ is —(CH₂)ₖN(Rₐ)R_{b}, in which
k is 0, 1 or 2;
Rₐ is hydrogen or an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group; and
R_{b} is a (C₃₋₈)cycloalkyl group, in which
(a) one of the carbon ring members of the (C₃₋₈)cycloalkyl moiety, which are different from the carbon ring member, to which the nitrogen atom carrying Rₐ is attached, is optionally replaced by a hetero ring member, selected from the group consisting of —O—, —S—, —S(═O)—, —S(═O)₂— and —N(R_{c})—, in which
R_{c} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group,
(b) the (C₃₋₈)cycloalkyl moiety is substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl-(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda*6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group, and
(c) the (C₃₋₈)cycloalkyl moiety is optionally substituted at two adjacent carbon ring members by two substituents, which form, together with the two adjacent carbon ring members, to which they are attached, a (C₃₋₈)cycloalkyl group, in which
(i) one of the carbon ring members of the (C₃₋₈)cycloalkyl group thus formed, which are different from the said two adjacent carbon ring members, to which the said two substituents are optionally attached, is optionally replaced by a hetero ring member, selected from the group consisting of —O—, —S—, —S(═O)—, —S(═O)₂— and —N(R_{d})—, in which
R_{d} is hydrogen or an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl or heteroaryl(C₁₋₄)alkyl group, and
(ii) the (C₃₋₈)cycloalkyl group thus formed is optionally substituted by 1 to 4 substituents, independently selected from the group consisting of halogen, cyano, oxo, hydroxy, (C₁₋₄)alkoxy, (C₁₋₄)alkoxy(C₁₋₄)alkoxy, (C₁₋₄)alkylthio, (C₁₋₄)alkylsulfinyl, (C₁₋₄)alkylsulfonyl, (C₁₋₄)alkylcarbonyl, (C₁₋₄)alkylcarbonyloxy, (C₁₋₄)alkoxycarbonyl, (C₁₋₄)alkoxycarbonyloxy and an optionally substituted (C₁₋₈)alkyl, (C₃₋₈)cycloalkyl, (C₃₋₈)cycloalkyl(C₁₋₄)alkyl, aryl, aryl(C₁₋₄)alkyl, heteroaryl, heteroaryl(C₁₋₄)alkyl, non-aromatic heterocyclyl, non-aromatic heterocyclyl(C₁₋₄)alkyl, chroman-4-yl, isochroman-4-yl, thiochroman-4-yl, isothiochroman-4-yl, 1,1-dioxo-1lambda*6*-thiochroman-4-yl, 2,2-dioxo-2lambda 6*-isothiochroman-4-yl, 1,2,3,4-tetrahydro-quinol-4-yl, 1,2,3,4-tetrahydro-isoquinol-4-yl, 1,2,3,4-tetrahydro-naphth-1-yl, 1,1-dioxo-1,2,3,4-tetrahydro-1lambda*6*-benzo[e][1,2]thiazin-4-yl, 2,2-dioxo-1,2,3,4-tetrahydro-2lambda*6*-benzo[c][1,2]thiazin-4-yl, 1,1-dioxo-3,4-dihydro-1H-1lambda*6*-benzo[c][1,2]oxathiin-4-yl, 2,2-dioxo-3,4-dihydro-2H-2lambda*6*-benzo[e][1,2]oxathiin-4-yl, 2,3,4,5-tetrahydro-benzo[b]oxepin-5-yl or 1,3,4,5-tetrahydro-benzo[c]oxepin-5-yl group;
R₂ is hydrogen or (C₁₋₈)alkyl;
R₃ is hydrogen, (C₁₋₈)alkyl or an optionally substituted (C₁₋₈)alkylOC(═O)NH, (C₃₋₈)cycloalkylOC(═O)NH, (C₃₋₈)cycloalkyl(C₁₋₄)alkylOC(═O)NH, aryl(C₁₋₄)alkylOC(═O)NH, heteroaryl(C₁₋₄)alkylOC(═O)NH, (C₁₋₄)alkylC(═O)NH, (C₃₋₈)cycloalkylC(═O)NH, arylC(═O)NH, aryl(C₁₋₄)alkylC(═O)NH, heteroarylC(═O)NH or heteroaryl(C₁₋₄)alkylC(═O)NH group;
U is a bond, CF₂, CF₂CF₂, CHF, CHFCHF, cycloprop-1,2-ylene, (C₁₋₃)alkylenoxy, (C₁₋₃)alkylenamino, (C₁₋₈)alkylene, NRₑ or an aromatic or heteroaromatic ring, which ring is optionally substituted with halogen, (C₁₋₈)alkoxy, hydroxy or (C₁₋₈)alkyl, whereby Z and V are in ortho- or meta-position to each other, wherein
Rₑ is hydrogen, (C₁₋₈)alkyl or (C₃₋₇)cycloalkyl;
V is CH═CH, cycloprop-1,2-ylene, CH₂CH(OH), CH(OH)CH₂ or CR_{f}R_{f}CR_{f}R_{f}, wherein each R_{f}, independently, is hydrogen, fluorine or (C₁₋₈)alkyl;
either
V₁ is hydrogen and
V₂ is hydroxy
or
V₁ and V₂ together are oxo;
W is (C₁₋₈)alkylene, O, S, S(═O)₂, C(═O), C(═O)O, OC(═O), N(R_{g})C(═O), C(═O)NR_{g} or NR_{g}, wherein
R_{g} is hydrogen or (C₁₋₈)alkyl;
X is an optionally substituted aromatic or heteroaromatic ring, whereby Y and C(═O)NR₂ are in meta-position to each other;
Y is a bond, O, S(═O)₂, S(═O)₂NRₕ, N(Rₕ)S(═O)₂, NRₕ, C(Rₕ)OH, C(═O)NRₕ, N(Rₕ)C(═O), C(═O)N(Rₕ)O or ON(Rₕ)C(═O), wherein
Rₕ is hydrogen, (C₁₋₈)alkyl or (C₃₋₈)cycloalkyl;
Z is O, CH₂, CF₂, CHF, CH═CH, cycloprop-1,2-ylene or a bond; and
n is 0 to 5,
the number of ring atoms included in the macrocyclic ring being 14, 15, 16 or 17,
in free base form or in acid addition salt form.

2. A process for the preparation of a compound as defined in claim 1 of the formula I, in free base form or in acid addition salt form, comprising the steps of
a) for the preparation of a compound of the formula I, in which R₁ is N(Rₐ)R_{b}, V₁ is hydrogen and V₂ is hydroxy, reaction of a compound of the formula 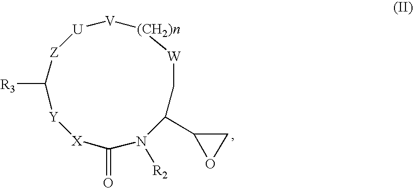
in which R₂, R₃, U, V, W, X, Y, Z and n are as defined for the formula I, with a compound of the formula HN(Rₐ)R_{b} (III), in which Rₐ and R_{b} are as defined for the formula I, or
b) cyclisation by metathesis of a suitable open chain-precursor compound, which carries, in each case, a carbon-carbon double bond at each of the two ends of the said open chain, in the presence of a catalyst, for instance a ruthenium, tungsten or molybdenum complex,
in each case optionally followed by reduction, oxidation or other functionalisation of the resulting compound and/or by cleavage of any protecting group(s) optionally present,
and of recovering the so obtainable compound of the formula I in free base form or in acid addition salt form.

3-4. (canceled)

5. A pharmaceutical composition, comprising:
the compound as defined in claim 1 of the formula I, in free base form or in pharmaceutically acceptable acid addition salt form, as active ingredient and
a pharmaceutical carrier or diluent.

6-7. (canceled)

8. A method for the treatment of neurological or vascular disorders related to beta-amyloid generation and/or aggregation in a subject in need of such treatment, comprising:
administering to such subject a therapeutically effective amount of a the compound as defined in claim 1 of the formula I, in free base form or in pharmaceutically acceptable acid addition salt form.

9. A combination, comprising:
a therapeutically effective amount of the compound as defined in claim 1 of the formula I, in free base form or in pharmaceutically acceptable acid addition salt form, and
a second drug substance, for simultaneous or sequential administration.
